SEQUENCE LISTING (1 sequence)

SEQ ID NO: 1 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

(19) United States
(12) Patent Application Publication
(10) Pub. No.: US 2026/0159564 A1
(43) Pub. Date: Jun. 11, 2026

(54) IL33 PROTEINS AND METHODS OF USE THEREOF

(71) Applicant: Memorial Sloan Kettering Cancer Center, New York, NY (US)

(72) Inventors: Vinod Balachandran, New York, NY (US); Taha Merghoub, Jersey City, NJ (US)

(21) Appl. No.: 18/707,578

(22) PCT Filed: Nov. 4, 2022
(86) PCT No.: PCT/US2022/079278

§ 371 Date: May 4, 2024

Related U.S. Application Data
(60) Provisional application No. 63/275,835, filed on Nov. 4, 2021.

(51) Int. Cl. C07K 14/54; A61K 38/00; A61P 35/00

(52) U.S. Cl. CPC C07K 14/54; A61P 35/00; A61K 38/00

(57) ABSTRACT

In some embodiments the present invention provides methods and compositions for inducing the formation of de novo tertiary lymphoid structures in subjects. In some embodiments such methods comprise administering an IL33 protein to a subject. In some embodiments the subjects have cancer. In some embodiments the subjects have a chronic inflammatory condition. In some embodiments the present invention provides methods for treating cancer. In some embodiments the present invention provides methods for treating chronic inflammatory conditions.

### CROSS-REFERENCE TO RELATED APPLICATIONS

This application claims the benefit of priority of U.S. Provisional Patent Application No. 63/275,835 filed on Nov. 4, 2021, the content of which is hereby incorporated by reference in its entirety.

### SEQUENCE LISTING

The instant application contains a Sequence Listing which has been submitted electronically in XML format and is hereby incorporated by reference in its entirety. Said XML copy, created on Nov. 4, 2022, is named ST26_MSKCC.055.WO1—Nov. 4, 2022, and is 2065 bytes in size.

### INCORPORATION BY REFERENCE

All references cited in this disclosure are hereby incorporated by reference in their entireties (numbers in parentheses or in superscript following text in this patent disclosure refer to the numbered references provided in the “Reference List” section of this patent specification). In addition, any manufacturers' instructions or catalogues for any products cited or mentioned herein are incorporated by reference. Documents incorporated by reference into this text, or any teachings therein, can be used in the practice of the present invention.

### BACKGROUND

Tertiary lymphoid structures (TLSs) are immune cell aggregates that form ectopically in inflamed tissues such as cancer. TLSs arise when inflammation induced-tissue damage stimulates TLS inducer cells to express lymphotoxin, the canonical lymphoneogenic protein, to recruit dendritic cells and B cells to prime antigen-specific T cells at inflammatory sites. The TLSs that are induced regulate immunity in chronic inflammation and in cancer. For example, in inflammatory bowel diseases, TLSs restrict pathogenic gut microbes. And in cancer, TLSs boost anti-tumor immunity, correlate with improved prognosis, and predict higher response to immune checkpoint inhibitors. Although TLSs critically modulate immune responses in cancer and inflammation, prior to the present invention the molecules and cells that induce TLS formation remained undefined.

### SUMMARY OF THE INVENTION

The present invention is based, in part, on a series of important discoveries that are described in more detail in the Examples section of this patent specification. In particular, it has now been discovered that interleukin-33 (IL33) can stimulate group 2 innate lymphoid cells (ILC2s) to induce the formation of de novo tertiary lymphoid structures (TLSs) in tumors-leading to inhibition of tumor growth, and in chronic inflammatory colitis—leading to increased survival.

Building on these discoveries, the present invention provides a variety of new and improved methods useful for therapeutic applications.

In particular, in some embodiments the present invention provides methods of inducing the formation of de novo tertiary lymphoid structures in subjects, such methods comprising administering to subjects in need thereof an effective amount of an IL33 protein or a pharmaceutical composition comprising an IL33 protein. In some such embodiments the subjects have cancer. In some such embodiments the subjects have a chronic inflammatory condition. In some embodiments the methods are used to treat cancer in the subjects. In some embodiments the methods are used treat a chronic inflammatory condition in the subjects.

These and other embodiments of the present invention are described in further detail in the Detailed Description, Examples, Claims, Figures and Brief Description of the Figures sections of this patent disclosure—each of which sections is intended to be read in conjunction with, and in the context of, all other sections of the present patent disclosure. Furthermore, one of skill in the art will recognize that the various embodiments of the present invention described herein can be combined in various ways, and that such combinations are within the scope of the present invention.

### BRIEF DESCRIPTION OF THE FIGURES

FIG. 1A-D. IL33 induces TLSs in pancreatic cancer. (FIG. 1A) Unbiased correlation of tumor mRNA gene expression to tertiary lymphoid structure (TLS) transcriptional signatures (Cabrita5, Coppola9, Gu-Trantien10) in human pancreatic ductal adenocarcinoma (PDAC) (top row). Correlation of tumor IL33 mRNA expression to TLS transcriptional signatures, and lymphotoxin beta (LTB) in human PDAC (a, bottom), breast cancer (b, top), and melanoma (FIG. 1B, bottom). n=number of tumors; all from The Cancer Genome Atlas (TCGA). (FIG. 1C) Representative immunohistochemistry and quantification of IL33+ cells in TLSs in human PDACs (15× magnification; inset, 50× magnification). (FIG. 1D) Representative hematoxylin and eosin (H&E), immunofluorescence (20× magnification) (top), and quantification (bottom) of intratumoral TLSs in PDAC mouse models with fewer (T cell low) or greater (T cell moderate) intratumoral T cells. Data were collected 2-3 weeks (cell lines 6694, 52), and 3-5 weeks (cell lines 6419, 50, 6499) after tumor implantation, pooled from two or more independent experiments with n≥2 mice per group; n=individual tumors from individual mice analyzed separately. Horizontal bars=median. P value by two-sided Pearson correlation (FIG. 1A, top), linear regression (FIG. 1A, bottom; FIG. 1B), and two-tailed Mann-Whitney test (FIG. 1C).

FIG. 2A-F. IL33 activates inflammatory ILC2s to express lymphotoxin. (FIG. 2A) Single-cell analysis of 1,668 purified tumor and draining lymph node (DLN) ILC2s from PDAC mice after 10 days of rIL33 treatment. UMAP plots (2 upper graphs) show single cells (dots) in a nonlinear representation of the top 15 principal components grouped by cluster (0, 1, 2, top left) and tissue (DLN, tumor, bottom left). Violin plots (8 lower graphs) of cluster-specific expression of select genes expressed in iILC2s. (FIG. 2B, D, E) Gating (FIG. 2B, D), frequency, lymphotoxin (LT, FIG. 2B), and ST2 (FIG. 2E) expression in KLRG1+ and KLRG1− ILC2s in rIL33-PDAC mice. (FIG. 2C) Gating, frequency, and number of KLRG1+ ILC2s in tumors and DLNs from rIL33-treated WT or ILC2-deficient PDAC mice. (FIG. 2F) LT mean fluorescence intensity (MFI) on blood and tumor KLRG1+ and KLRG1− ILC2s in cancer patients. Histogram=tumor ILC2s; lines=paired data within samples; n=number of samples per category. (FIG. 2A-E) Data were collected 10 days (FIG. 2A), 5 weeks (FIG. 2B, C, D), or 4-5 weeks (FIG. 2E) after tumor implantation, pooled from two or more independent experiments with n≥2 mice per group; each point indicates one mouse analyzed separately. n=number of tumors from individual mice. MFI=mean fluorescence intensity. Horizontal bars=median. P value by two-way ANOVA with Holm (FIG. 2A), and Tukey's multiple comparison (FIG. 2B) tests, and two-tailed Mann-Whitney test (FIG. 2C-F).

FIG. 3A-F: Inflammatory ILC2s migrate to control primary and distant tumors. (FIG. 3A-C) CD45.2 donor and CD45.1 recipient parabiotic mice were implanted with PDACs in recipient pancreata (FIG. 3A), or in donor pancreata and recipient subcutaneous (SQ) tissue (FIG. 3B). Donors were treated with vehicle, rIL33 (FIG. 3A-C), or rIL25 (FIGS. 3B & C). Gating and frequency of donor-derived ILC2s in recipient pancreatic (FIG. 3A) or SQ (FIGS. 3B & C) PDACs. (FIG. 3D-F) SQ PDAC growth, KLRG1+ ILC2 frequency, and number in WT (FIG. 3D), Il1rl1−/− (ST2-deficient) (FIG. 3E), or ILC2-deficient (FIG. 3F) mice with SQ PDAC alone, or SQ and pancreatic PDACs. Data were collected at 2 (FIG. 3A-C), 5 (FIGS. 3D & F), or 3 (FIG. 3E) weeks after tumor implantation, pooled from two or more independent experiments with n≥3 mice per group; each point indicates one mouse analyzed separately. n=number of tumors from individual mice. Horizontal bars=median. P value by two-way ANOVA with Kruskal-Wallis (FIG. 3B-D frequency, number) or Sidak's (FIG. 3D-F, tumor weight) multiple comparison tests, and two-tailed Mann-Whitney test (FIG. 3A, D-F).

FIG. 4. A-I: Inflammatory ILC2s utilize lymphotoxin to induce TLS and can be harnessed for cancer immunotherapy. (FIG. 4A) Representative H&E and TLS number in tumors of rIL33-treated wild-type (WT) and ILC2-deficient pancreatic PDAC mice. (FIG. 4B) Representative H&E, and TLS number (in pancreatic PDAC), tumor growth, and KLRG1+ ILC2 frequency in rIL33-treated WT and Ltbr−/− pancreatic and SQ PDAC mice. (FIGS. 4C & H) Tumor KLRG1+ ILC2s were sort-purified from rIL33-treated WT or Ltb−/− pancreatic PDAC mice and transferred to ILC2-deficient pancreatic PDAC recipients. (FIG. 4C) KLRG1+ ILC2 frequency and number, TLS number, and tumor growth in recipient PDACs. (FIGS. 4 D, E) Gating, LTbR expression (FIG. 4D), and phenotype (FIG. 4E) of intratumoral LTbR+IL33+ cells in rIL33-treated pancreatic PDAC mice. (FIG. 4F) Intratumoral LTbR+ cells were sort-purified from tumors of rIL33-treated WT or Il33−/− pancreatic PDAC mice and co-implanted with tumors into pancreata of Il33−/− recipients (top). Intratumoral KLRG1+ ILC2 LT expression, TLS number, and tumor weight (bottom). (FIG. 4G) Intratumoral IL33+ CXCL13+ cell frequency in rIL33-treated WT and Ltbr−/− pancreatic PDAC mice. (FIG. 4H) Gating, CXCL13+ cell frequency, and mean fluorescence intensity (MFI) in IL33+ LTbR+ cells in recipient pancreatic PDACs (experimental schema in FIG. 4C). (FIG. 4I) IL33 expression in purified WT or Ltbr−/− myeloid cells (left), or WT myeloid cells treated with agonistic LTbR-Ig in vitro. Data in (FIG. 4A-I) were collected at 4 weeks post implantation (FIG. 4A, B, D, E, G), 2 weeks post cell transfer (FIGS. 4C, F, H), and 24 hours post-stimulation (FIG. 4I), pooled from two or more independent experiments with n≥2 mice per group; each point indicates one mouse analyzed separately. n=number of tumors from individual mice. Horizontal bars=median. P value by two-tailed Mann-Whitney test (FIG. 4A, D-I), two-way ANOVA with Sidak's multiple comparison (FIG. 4B, tumor volume), and one-way ANOVA with Kruskal Wallis multiple comparison test.

FIG. 5A-B. IL33 expression correlates with TLS signatures and IL33-expressing cells are present in human PDAC. (FIG. 5A) Correlation of intratumoral IL33 mRNA expression to three TLS transcriptional signatures (Cabrita5, Coppola9, Gu-Trantien10), and LTB mRNA expression in prospectively collected (MSK, top) and previously published (ICGC, bottom) human PDAC cohorts. (FIG. 4B) IL33 immunohistochemistry in human PDACs with TLSs. n=number of tumors. P value by linear regression.

FIG. 6. Intratumoral T cell infiltrates vary in PDAC mouse models. Frequency of CD4+ and CD8+ T cells in T cell low and T cell moderate pancreatic PDAC mouse models. n=number of tumors from individual mice. Data collected at 2-3 (6694, 52), 3-4 (6419), and 4-6 (6499, 50, 4662) weeks after tumor implantation, pooled from two or more independent experiments with n≥3 mice per group; each point indicates one mouse analyzed separately. Horizontal bars=median. P value by two-tailed Mann-Whitney test.

FIG. 7A-F: IL33 expands iILC2s in blood and PDACs in mice. (FIG. 7A) Gating strategy to identify KLRG1+ ILC2s in mice. (FIG. 7B) Gating strategy to detect lymphotoxin (LT) expression on ILC2s. LT expression on KLRG1+ ILC2s purified from wild-type (WT) and Ltb−/− mice 4 weeks after tumor implantation; FMO (fluorescence minus one)=negative controls; MFI=mean fluorescence intensity. (FIG. 7C, D) Single-cell analysis of 1,668 purified tumor and draining lymph node (DLN) ILC2s from pancreatic PDAC mice after 10 days of rIL33 treatment, depicting ILC transcription factors (FIG. 7C), surface markers and cytokines (FIG. 7D). (FIG. 7E) Blood and DLN KLRG1+ TLC2 frequencies in a T cell moderate pancreatic PDAC mouse model (cell line 4662) collected 5 (blood) or 32 (DLN) days after tumor implantation. (FIG. 7F) Intratumoral KLRG1+ TLC2 frequency in T cell low (left) and T cell moderate (right) pancreatic PDAC mouse models treated with vehicle or rIL33 and collected at the timepoints described in FIG. 6. Data were pooled from two or more independent experiments with n≥2 mice per group; each point indicates one mouse analyzed separately. n=number of tumors from individual mice. Horizontal bars=median. P value by two-tailed Mann-Whitney test (FIG. 7B, E, F).

FIG. 8 iILC2s infiltrate human tumors. Gating strategy to identify inflammatory ILC2s (iILC2s defined as KLRG1+ ILC2s) in humans. iILC2s in a human primary PDAC tumor is shown. Quantification of iILC2s in human tumors is shown in FIG. 2F.

FIG. 9A-E. IL33 does not induce non-ILC2s to migrate in parabiotic PDAC mice. (FIG. 9A) Experimental schema. (FIG. 9B) Gating and quantification of donor (CD45.2) and recipient (CD45.1) leukocytes in recipient blood. (FIG. 9C) Gating and quantification of donor-derived KLRG1+ and KLRG1− ILC2s in recipient blood. (FIG. 9D) Gating and quantification of donor-derived non-ILCs in recipient blood and pancreatic PDACs. (FIG. 9E) Gating strategy to identify donor-derived KLRG1+ and KLRG1− ILC2s in recipient PDACs. Data were collected 7 (blood) or 14 (tumor) days after tumor implantation. Data were pooled from two or more independent experiments with n≥3 mice per group; each point indicates one mouse analyzed separately. n=number of tumors from individual mice. Horizontal bars=median. P value by two-tailed Mann-Whitney test (FIG. 9B, D).

FIG. 10A-D. IL25 does not induce ILC2s to migrate to tumors in parabiotic PDAC mice. (FIG. 10A) Experimental schema. (FIG. 10B) Gating and quantification of donor (CD45.2) ILC2s in donor pancreatic PDACs. (FIGS. 10C, D) Quantification of donor-derived KLRG1+ ILC2s (FIG. 10C) and non-ILCs (FIG. 10D) in recipient blood (FIGS. 10C, D) and SQ PDACs (FIG. 10D). Data were collected 14 (FIG. 10B), 5-9 (FIG. 10C), or 7 (FIG. 10D) days after tumor implantation and pooled from two or more independent experiments with n≥3 mice per group; each point indicates one mouse analyzed separately. n=number of tumors from individual mice. Horizontal bars=medians. P value by two-way ANOVA with Sidak's multiple comparison test (FIG. 10B-D). individual mice. Horizontal bars=medians. P value by two-way ANOVA with Sidak's multiple comparison test (FIG. 10B-D).

FIG. 11A-B. iILC2s and LTbR+ myeloid cells can be purified from murine PDACs. Representative pre- and post-sort purity of KLRG1+ ILC2s (FIG. 11A) and LTbR+ myeloid cells (FIG. 11B) from murine PDAC.

FIG. 12. Left: Schematic of dextran sodium sulfate (DSS) colitis model of inflammation-induced colonic tertiary lymphoid structures (TLS). Wild-type (WT) or IL33−/− mice were treated with 3% DSS in drinking water for 7 days and allowed to recover for 14 days (no DSS exposure). TLS in the colon were quantified. Right: Schematic of αLTβR agonistic antibody therapy in a mouse PDAC model. WT or IL33−/− orthotopic PDAC mice were treated with αLTβR agonistic antibody every 3 days. TLS in PDAC tumors were analyzed 14 days after implantation. Data were pooled from of two independent experiments with n=5 mice per group; n=individual tumors from individual mice analyzed separately. Horizontal bars=media. P values by 2-way ANOVA with Tukey's multiple comparison tests.

FIG. 13. Experimental schematic (top). Wild-type (WT) mice were treated with 3% dextran sodium sulfate (DSS) in drinking water and rIL33 (500 ng) daily for 7 days, after which they were allowed to recover for 7 days (no DSS exposure; rIL33 3 times/week), and survival was assessed (bottom). n=individual mice. P value by 2-way log-rank test.

### DETAILED DESCRIPTION

Some of the embodiments of the present invention are described in the “Summary of the Invention,” “Examples,” “Brief Description of the Figures,” and “Figures” sections of this patent disclosure. This Detailed Description section provides certain additional embodiments and certain additional description and details relating to embodiments described elsewhere herein, and is intended to be read in conjunction with all other sections of the present patent disclosure.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Ausubel et al. eds. (2015) Current Protocols in Molecular Biology (John Wiley and Sons); Greenfield, ed. (2013) Antibodies: A Laboratory Manual (2nd ed., Cold Spring Harbor Press); Green and Sambrook, eds. (2012), Molecular Cloning: A Laboratory Manual (4th ed., Cold Spring Harbor Laboratory Press); Krebs et al., eds. (2012) Lewin's Genes XI (11th ed., Jones & Bartlett Learning); Freshney (2010) Culture Of Animal Cells (6th ed., Wiley); Weir and Blackwell, eds., (1996) Handbook Of Experimental Immunology, Volumes I-IV (5th ed., Wiley-Blackwell); Borrebaeck, ed. (1995) Antibody Engineering (2nd ed., Oxford Univ. Press); Glover and Hames, eds., (1995) DNA Cloning: A Practical Approach, Volumes I and II (2nd ed., IRL Press); Rees et al., eds. (1993) Protein Engineering: A Practical Approach (1st ed., IRL Press); Mayer and Walker, eds. (1987) Immunochemical Methods In Cell And Molecular Biology (Academic Press, London); Nisonoff (1984) Introduction to Molecular Immunology (2nd ed., Sinauer Associates, Inc.); and Steward (1984) Antibodies: Their Structure and Function (1st ed., Springer Netherlands).

In order that the present invention can be more readily understood, certain terms are defined herein. Additional definitions are set forth throughout the disclosure. Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

### Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range.

Where a numeric term is preceded by “about” or “approximately,” the term includes the stated number and values ±10% of the stated number.

Numbers in parentheses or superscript following text in this patent disclosure refer to the numbered references provided in the “Reference List” section of this patent disclosure.

Wherever embodiments are described with the language “comprising,” otherwise analogous embodiments described in terms of “consisting of” and/or “consisting essentially of” are included.

Amino acids are referred to herein by their commonly known three-letter symbols or by the one-letter symbols recommended by the IUPAC-IUB Biochemical Nomenclature Commission. Nucleotides, likewise, are referred to by their commonly accepted single-letter codes.

As used herein the abbreviation “IL33” refers to interleukin 33.

As used herein the abbreviation “ILC2” refers to group 2 innate lymphoid cells.

As used herein the abbreviation “IP” or “i.p.” refers to intraperitoneal. It is common to administer agents to mice via an IP route, which is considered to be analogous to administering an agent to a human subject by an IV route.

As used herein the abbreviation “IT” refers to intratumoral. For example, a drug injected directly into a tumor is delivered intratumorally.

As used herein the abbreviation “IV” refers to intravenous.

As used herein the abbreviation “PDAC” refers to pancreatic ductal adenocarcinoma.

As used herein the abbreviation “TILC2” refers to tumor ILC2s. It should be noted that all of the embodiments described herein that refer to ILC2s are also intended to encompass TILC2s, and that for all of the methods described herein as involving ILC2s, alternatives that are directed to TILCs-specifically are also contemplated by the present invention.

As used herein the abbreviation “TLS refers to tertiary lymphoid structures. As used herein the abbreviation PD-1” refers to Programmed Death 1, which is also known as Programmed Death Protein 1 or Programmed Cell Death Protein 1.

As used herein the abbreviation PD-L1 refers to Programmed Cell Death Ligand 1—which is a ligand for PD-1.

The terms “identical” or “percent identity” in the context of two or more amino acid sequences, refer to two or more amino acid sequences or subsequences that are the same (identical) or have a specified percentage of amino acid residues that are the same (percent identity), when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

As used herein the term “subject” refers to an individual for whom treatment using a composition or method of the present invention may be contemplated. In some embodiments a subject is a mammalian subject, such as a human, domestic pet, animal used in agriculture or food production, sports animal, zoo animal, and the like. In some embodiments the subject is a non-human primate. In some embodiments the subject is a rodent. In some embodiments the subject is a human.

As used herein the term “vector” refers to a construct for delivery of a nucleic acid molecule to a host cell. Examples of vectors include, but are not limited to, viruses, viral-derived vectors, naked DNA or RNA vectors, plasmid vectors, cosmid vectors, phage vectors, and the like. In some embodiments a vector may be an “expression vector” that is capable of delivering a nucleic acid molecule to a host cell and that also contains elements required for expression of the nucleic acid molecule in the host cell.

Other terms are defined elsewhere in this patent disclosure, or else are used in accordance with their usual meaning in the art.

### Active Agents

The methods and compositions of the present invention involve active agents that are IL33 proteins.

Several IL33 proteins are known in the art, including several naturally occurring, non-naturally occurring, and/or recombinant IL33 proteins. Examples of IL33 proteins that are known in the art include, but are not limited to, recombinant murine IL33 (commercially available from R&D Systems, in carrier-containing or carrier-free forms), murine IL33 having the amino acid sequence set forth in UniProtKB/Swiss-Prot: Q8BVZ5.1, recombinant human IL33 (commercially available from R&D Systems, in carrier-containing or carrier-free forms), and human IL33 having the amino acid sequence set forth in UniProtKB/Swiss-Prot: 095760.1. Any IL33 protein known in the art can be used in the methods or compositions of the present invention, provided that such IL33 protein has at least one of the following properties: (a) the ability to bind to the IL33 receptor ST2, (b) the ability to activate the IL33 receptor ST2, or (c) the ability to induce the formation of TLSs.

The full-length wild-type version of the human IL33 is 270 amino acids long (aa 1-270). Mature versions of the wild-type human IL33 protein are generated by proteolytic cleavage of the full-length protein, and include a mature version that consists of the last/C-terminal 159 amino acids of the full-length protein—i.e., aa 112-270. As used herein references to “rIL33” refer to the mature version of wild-type human IL33 consisting of amino acid residues 112-270 of full length human IL33. The amino acid sequence of rIL33 is known in the art, and is also provided herein as SEQ ID NO. 1 for convenience. In some embodiments the IL33 protein used in the methods or compositions of the present invention is rIL33.

TABLE 1

| | Description/ | |
|---|---|---|
| SEQ ID NO. | Name | Amino Acid Sequence |
| SEQ ID NO. 1 | rIL-33 | SITGISPITEYLASLSTYNDQSITFALEDESYEI |
| | (aas 112-270) | YVEDLKKDEKKDKVLLSYYESQHPSNESGD |
| | | GVDGKMLMVTLSPTKDFWLHANNKEHSVE |
| | | LHKCEKPLPDQAFFVLHNMHSNCVSFECKT |
| | | DPGVFIGVKDNHLALIKVDSSENLCTENILF |
| | | KLSET |

In addition to the specific examples of IL33 proteins provided herein (e.g., rIL33), IL33 proteins that are modified as compared to those IL33 proteins, or that have amino acid sequences that vary as compared to those IL33 proteins, can also be used in the methods and compositions of the present invention, provided that such IL33 proteins have at least one of the following properties: (a) the ability to bind to the IL33 receptor ST2, (b) the ability to activate the IL33 receptor ST2, or (c) the ability to induce the formation of TLSs.

In some embodiments, the IL33 proteins used comprise an IL33 sequence and one or more additional moieties.

In some embodiments such additional moieties are protein/peptide moieties. In some embodiments such additional moieties are non-proteinaceous chemical moieties. In some embodiments such additional moieties are N-terminal to the IL33 sequence. In some embodiments such additional moieties are C-terminal to the IL33 sequence. In some embodiments such additional moieties are at both the N- and C-terminal ends of the IL33 sequence.

In some embodiments such additional moieties facilitate and/or improve the production, purification, stability half-life, bioavailability, formulation, ST2-binding affinity, or any other therapeutically desirable properties of the IL33 sequence.

For example, in some embodiments such additional moieties are tags useful for detection and/or purification. Exemplary tags include, but are not limited to, Strep tags, Strep II tags, FLAG tags, glutathione S-transferase (GST) tags, green fluorescent protein (GFP) tags, hemagglutinin A (HA) tags, histidine (His) tags, luciferase tags, maltose-binding protein (MBP) tags, c-Myc tags, protein A tags, protein G tags, and the like.

In some embodiments such additional moieties are leader sequences, precursor polypeptide sequences, secretion signals, and/or localization signals.

In some embodiments modified IL33 proteins according to the present invention may comprise one or more PEG molecules—i.e., they may be pegylated.

In some embodiments modified IL33 proteins according to the present invention may comprise an immunoglobulin Fc domain.

In some embodiments modified IL33 proteins according to the present invention may comprise an albumin-binding domain.

Any additional moieties known in the art can be used provided that the modified IL33 protein (i.e., with the additional moiety) has at least one of the following properties: (a) the ability to bind to the IL33 receptor ST2, (b) the ability to activate the IL33 receptor ST2, or (c) the ability to induce the formation of TLSs.

In some embodiments such additional moieties are directly attached (either covalently or non-covalently) to the IL33 amino acid sequences. In some embodiments additional moieties are indirectly attached to the IL33 amino acid sequences via a linker, such as a peptide linker. In each case, the additional moieties may be coupled to the peptides (whether directly or indirectly) using any suitable means known in the art, including by chemical conjugation or, in the case of amino acid or peptide or protein moieties, by expression as a fusion protein.

IL33 proteins as described herein can be prepared using any suitable means known in the art. For example, IL33 proteins can be prepared using recombinant DNA methods. For example, polynucleotides encoding the IL33 proteins can be cloned into suitable expression vectors.

Transfection of host cells with the expression vector results in generation of the engineered IL33 proteins by the host cells. In some embodiments the IL33 is synthetically produced.

IL33 proteins used in the methods of the present invention may be provided in a composition, for example in a pharmaceutical composition that comprise the IL33 protein. As used herein the term “pharmaceutical composition” refers to a preparation that is in such form as to permit the biological activity of the active agent (e.g., an IL33 protein) to be effective and which contains no additional components that are unacceptably toxic to a subject to whom the composition may be administered. Such compositions may be sterile. Typically, such compositions comprise a pharmaceutically acceptable excipient. Examples of pharmaceutically acceptable excipients include, but are not limited to, water, physiological saline, salts, buffers (e.g., acetate, phosphate or citrate buffers), surfactants (e.g., polysorbate), stabilizing agents (e.g., human albumin), solubilizing agents, dispersing agents, preservative/es (e.g., benzyl alcohol), and the like.

### Methods

In some embodiments the present invention provides methods of inducing the formation of de novo tertiary lymphoid structures in a subject. Such methods comprise administering an effective amount of an IL33 protein (or a composition, such as a pharmaceutical composition, comprising an IL33 protein) to a subject.

In some embodiments the present invention provides various methods of treatment. For example, in some embodiments the present invention provides methods of treatment that comprise administering an effective amount of an IL33 protein to subject.

As used herein, the terms “treat,” “treating,” and “treatment” encompass achieving, and/or performing a method that achieves, a detectable improvement in one or more clinical indicators or symptoms associated with a given disease, syndrome or condition (the terms “disease,” “syndrome” and “condition” may be used interchangeably herein). For example, in the case where the disease or condition is cancer, such terms include, but are not limited to, reducing the rate of growth of a tumor (or of tumor cells), halting the growth of a tumor (or of tumor cells), causing regression of a tumor (or of tumor cells), reducing the size of a tumor (for example as measured in terms of tumor volume or tumor mass), reducing the grade of a tumor, eliminating a tumor (or tumor cells), preventing, delaying, or slowing recurrence (rebound) of a tumor, improving symptoms associated with a tumor, improving survival from a tumor, inhibiting or reducing spreading of a tumor (e.g. metastases), and the like. In each of the embodiments described herein that refer to a method of treatment, a method of achieving any one or more of the specific parameters listed above is also contemplated. For example, for each of the embodiments described herein that refers to a method of treating a tumor, the following methods are also contemplated and are intended and fall within the scope of the invention: (a) a method of reducing the rate of growth of the tumor, (b) a method of halting the growth of the tumor, (c) a method of causing regression of the tumor, (d) a method of reducing the size of the tumor, (e) a method of reducing the grade of the tumor, (f) a method of eliminating the tumor, (g) a method of preventing, delaying, or slowing recurrence (rebound) of the tumor, (h) a method of improving symptoms of the tumor, (i) a method of improving survival from the tumor, and (j) a method of inhibiting or reducing spreading of the tumor (e.g. metastasis). Similarly, in the case where the disease or condition is a chronic inflammatory disease, such treatment terms include, but are not limited to, reducing the severity of the disease, reducing the duration of the disease, improving survival from the disease, improving one or more symptoms of the disease, and the like.

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In some embodiments the subjects are human.

In some embodiments the subjects have cancer. In some embodiments the subjects have pancreatic cancer. In some embodiments the subjects have pancreatic ductal adenocarcinoma (PDAC). In some embodiments the subjects have breast cancer. In some embodiments the subjects have melanoma. In some embodiments the present methods and compositions can be used to treat a tumor or cancer in a subject in need thereof. In some embodiments the present methods and compositions can be used to treat a pancreatic tumor in a subject in need thereof (i.e., in a subject with pancreatic cancer). In some embodiments the present methods and compositions can be used to treat pancreatic ductal adenocarcinoma (PDAC) in a subject in need thereof (i.e., in a subject with PDAC). In some embodiments the present methods and compositions can be used to treat breast cancer in a subject in need thereof (i.e., in a subject with breast cancer). In some embodiments the present methods and compositions can be used to treat melanoma in a subject in need thereof (i.e., in a subject with melanoma). In some embodiments the subject has a tumor that is resistant to treatment using other methodologies and/or compositions. As used herein, the terms “resistant” and “resistance” are used consistent with their normal usage in the art and consistent with the understanding of those term by physicians who treat cancer (e.g., oncologists). For example, consistent with its usual meaning in the art, a tumor or a subject may be considered “resistant” to a certain treatment method or treatment with a certain agent (or combination of agents), if, despite using that method or administering that agent (or combination of agents), a subject's tumor (or tumor cells) grows, and/or progresses, and/or spreads, and/or metastasizes, and/or recurs. In some instances, a tumor may initially be sensitive to treatment with a certain method or agent (or combination of agents), but later became resistant to such treatment.

In some embodiments the present methods and compositions can be used to treat a PD-1/PD-L1 inhibitor resistant tumor/cancer in a subject in need thereof (i.e., in a subject with a PD-1 and/or PD-L1 inhibitor resistant tumor/cancer). In some embodiments the present methods and compositions can be used to treat PD-1/PD-L1 inhibitor resistant PDAC in a subject in need thereof (i.e., in a subject with PD-1 and/or PD-L1 inhibitor resistant PDAC).

In some embodiments the subject has a tumor/cancer that has recurred following a prior treatment with other compositions or methods, including, but not limited to, chemotherapy, radiation therapy, or surgical resection, or any combination thereof. In some embodiments the subject has a pancreatic tumor that has not previously been treated.

In some embodiments the subjects have a chronic inflammatory condition. In some embodiments the subjects have a chronic gastrointestinal inflammatory condition. In some embodiments the subjects have a chronic gastrointestinal inflammatory condition selected from colitis, ulcerative colitis, inflammatory bowel disease, irritable bowel syndrome and Crohn's disease.

In some embodiments the present methods and compositions can be used to treat a chronic inflammatory condition in a subject. In some embodiments the present methods and compositions can be used to treat a chronic gastrointestinal inflammatory condition. In some embodiments the present methods and compositions can be used to treat a chronic gastrointestinal inflammatory condition selected from colitis, ulcerative colitis, inflammatory bowel disease, irritable bowel syndrome and Crohn's disease.

As used herein the term “effective amount” refers to an amount of an active agent (i.e., an IL33 protein) or pharmaceutical composition as described herein that is sufficient to induce the formation of tertiary lymphoid structures in a subject. An appropriate “effective” amount in any individual case may be determined using standard techniques known in the art, such as dose escalation studies, and may be determined taking into account such factors as the desired route of administration (e.g., systemic vs. intratumoral), desired frequency of dosing, etc. Furthermore, an “effective amount” may be determined in the context of any co-administration method to be used. One of skill in the art can readily perform such dosing studies (whether using single agents or combinations of agents) to determine appropriate doses to use, for example using assays such as those described in the Examples section of this patent application—which involve administration of the agents described herein to subjects (such as animal subjects routinely used in the pharmaceutical sciences for performing dosing studies).

For example, in some embodiments the dose of an active agent of the invention may be calculated based on studies in humans or other mammals carried out to determine efficacy and/or effective amounts of the active agent. The dose may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In some embodiments suitable doses of the various active agents described herein can be determined by performing dosing studies of the type that are standard in the art, such as dose escalation studies, for example using the dosages shown to be effective in mice in the Examples section of this patent application as a starting point.

In some embodiments one or more of the active agents is used at approximately its maximum tolerated dose, for example as determined in phase I clinical trials and/or in dose escalation studies. In some embodiments one or more of the active agents is used at about 90% of its maximum tolerated dose. In some embodiments one or more of the active agents is used at about 80% of its maximum tolerated dose. In some embodiments one or more of the active agents is used at about 70% of its maximum tolerated dose. In some embodiments one or more of the active agents is used at about 60% of its maximum tolerated dose. In some embodiments one or more of the active agents is used at about 50% of its maximum tolerated dose. In some embodiments one or more of the active agents is used at about 50% of its maximum tolerated dose. In some embodiments one or more of the active agents is used at about 40% of its maximum tolerated dose. In some embodiments one or more of the active agents is used at about 30% of its maximum tolerated dose.

In carrying out the methods described herein, any suitable method or route of administration can be used to administer the active agents (i.e., IL33 proteins) or pharmaceutical compositions to subjects. In some embodiments systemic administration may be employed, for example, oral or intravenous (IV) administration, or any other suitable method or route of systemic administration known in the art. In some embodiments intratumoral (IT) delivery may be employed. In some embodiments intraperitoneal (IP) delivery may be employed. For example, the active agents described herein may be administered either systemically or locally by injection, by infusion through a catheter, using an implantable drug delivery device, or by any other means known in the art.

In certain embodiments the pharmaceutical compositions and methods provided herein may be employed together with other pharmaceutical compositions and methods known to be useful for cancer therapy, including, but not limited to, surgical methods (e.g., for tumor resection), radiation therapy methods, treatment with chemotherapeutic agents, treatment with antiangiogenic agents, treatment with tyrosine kinase inhibitors or treatment with immune checkpoint inhibitors. Similarly, in certain embodiments the methods provided herein may be employed together with procedures used to monitor disease status/progression, such as biopsy methods and diagnostic methods (e.g., MRI methods or other imaging methods).

For example, in some embodiments the methods described herein may be performed prior to performing surgical resection of a tumor, for example to shrink a tumor prior to surgical resection. In other embodiments the methods described herein may be performed both before and after performing surgical resection of a tumor.

In certain embodiments the pharmaceutical compositions and methods provided herein may be employed together with other pharmaceutical compositions and methods known to be useful for the treatment of chronic inflammatory conditions.

The invention is further described by, and understood with reference to, the following non-limiting Examples, as well as the Figures referred to therein.

### Examples

### IL33-Activated Migratory ILC2s Induce Tertiary Lymphoid Structures in Pancreatic Cancer

Numbers in parentheses at the end of sentences or following various statements herein refer to the numbered references in the Reference List section of this patent application.

Tertiary lymphoid structures (TLSs) are specialized de novo lymphoid organs that arise ectopically in injured tissues to regulate immunity in chronic inflammation and cancer (1). Yet, although TLSs are ubiquitous structures that critically bolster host defense in chronic tissue injury, the molecules and cells that initiate TLSs remain elusive. Here, we discover that interleukin-33 (IL33), the alarmin released by damaged tissues (2), stimulates group 2 innate lymphoid cells (ILC2s) to induce TLSs in cancer. We identify IL33 as among the genes most highly correlated to TLS transcriptional signatures in human pancreatic ductal adenocarcinoma (PDAC), breast cancer, and melanoma. Furthermore, in human cancers, we discover rare IL33-expressing cells within TLSs, and IL33-responsive inflammatory ILC2s (iILC2s) expressing lymphotoxin (LT), the TLS-inducing cytokine (3). In mice, IL33 unexpectedly induces iILC2s to migrate into PDACs to generate de novo TLSs. Mechanistically, IL33 stimulates iILC2s to express LT that activates novel LTb receptor (LTbR)+ myeloid cells to produce the canonical lymphoid chemokine CXCL13 in tumors. iILC2s also utilize LT to induce LTbR+ myeloid cells to produce IL33 that reciprocally induces iILC2s to express LT in a feedback loop. We also show that human recombinant IL33 (H-rIL33) expands iILC2s and TLSs to control PDAC in mice, and expands LT+ ILC2s in humans. Thus, in sum, we discover a previously unknown lymphoneogenic pathway—initiated by IL33, induced by ILC2s, and organized by myeloid cells. Furthermore, we harness this pathway for cancer immunotherapy, identifying IL33 and ILC2s as new targets that can modulate TLSs to treat cancer and other chronic tissue pathologies. We also identify a previously unrecognized function for ILC2s to initiate ectopic lymphoneogenesis in tissues.

Secondary lymphoid organs (SLOs) are key structures in mammals (4) that organize immune cells at prenatally determined strategic anatomic sites. SLOs drain antigens from specific tissues, and co-localize cells that sample these antigens with cells that execute ensuing immune responses, to thereby efficiently patrol tissues and clear threats. However, when threats persist, as in chronic infections or tumors, hosts must develop lymphoid organs ectopically directly within tissues to boost both the intensity and proximity of the immune response. These ectopic lymphoid organs, termed tertiary lymphoid structures (TLSs), are a ubiquitous feature in chronic tissue pathologies, (1) including infection, inflammation, and neoplasia, and serve to regulate immunity. In cancer, hosts ostensibly develop TLSs in any tumor that arises in a tissue (1), and these TLSs boost both endogenous (1) and therapeutic (5-7) anti-tumor immunity in humans and mice. Therefore, developing new cancer immunotherapies to induce TLSs as a means to augment anti-tumor immunity is an attractive goal, as most human tumors have few baseline immune cells (“cold” tumors) that effectively render them resistant to current immunotherapies.

### IL33 Induces Tertiary Lymphoid Structures in Pancreatic Cancer

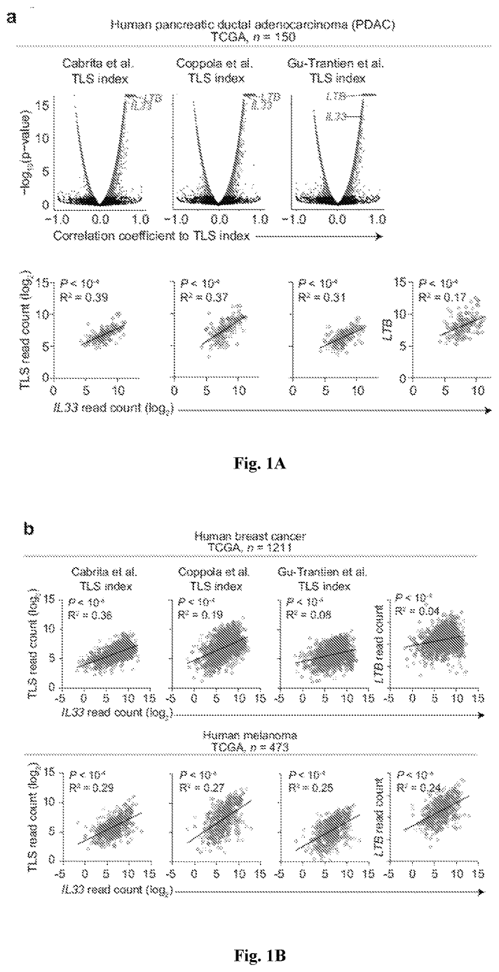

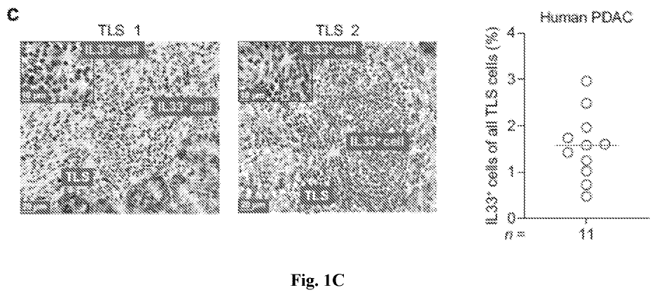

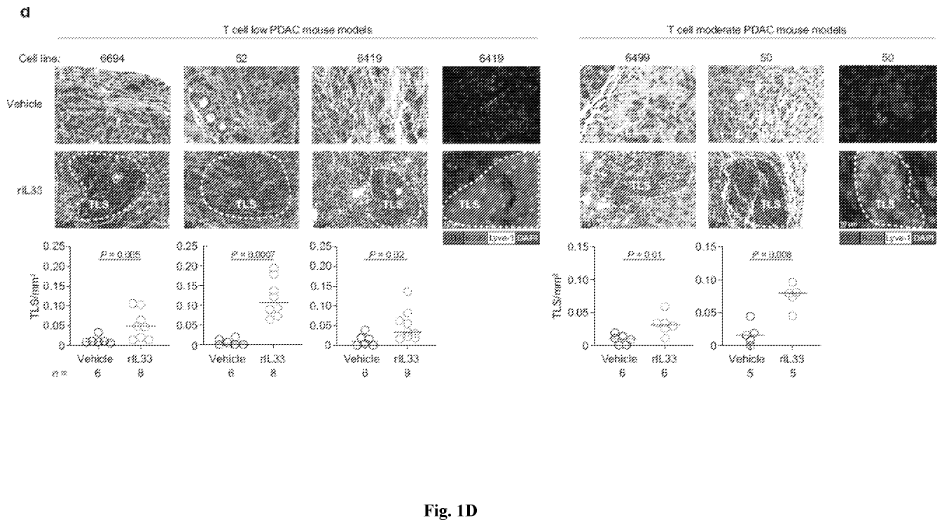

To identify candidate signals that induce TLSs in tumors, we searched in The Cancer Genome Atlas (TCGA) (8) for genes whose expression positively correlated to TLS transcriptional signatures (5,9,10) in pancreatic ductal adenocarcinoma (PDAC), a classic cold tumor where higher intratumoral TLS density boosts immunity (1)1 and correlates with longer survival (12). As TLSs can be identified by their unique inducing chemokines (including the canonical CXCL1313), as well as populating cells (activated T cells, B cells, dendritic cells, and myeloid cells (1)), we selected three largely non-overlapping transcriptional signatures that identify TLSs based on such chemokines (9), cells (10), and other immunotherapy-promoting factors (5). Interestingly, we found that interleukin-33 (IL33), which encodes an alarmin rapidly released extracellularly by damaged tissues (2), was among the genes most highly correlated to expression of all three TLS signatures, and to lymphotoxin beta (LTB), which encodes the canonical lymphoid tissue (14) and TLS-inducing cytokine (3) LTb (FIG. 1A). To confirm this, we tested if IL33 correlated to TLS transcriptional signatures and LTB in a second prospectively collected cohort (FIG. 5A, top), and a third independently published(15) cohort (FIG. 5A, bottom) of PDAC patients. In both cohorts, we found IL33 expression strongly correlated to expression of all three TLS transcriptional signatures, and to LTB (FIG. 5A). To understand whether this finding extended to other human tumors, we examined IL33, TLS transcriptional signatures, and LTB expression in another immunologically cold (breast), as well as an immunologically “hot” (more baseline immune cells) tumor (melanoma) (FIG. 1B). As in PDAC, we found IL33 expression in tumors strongly correlated to TLS transcriptional signatures and LTB expression in both tumor types (FIG. 1B). We next searched for IL33+ cells within TLSs in human PDAC, to examine if IL33 was in fact expressed in TLSs. Consistently, we found IL33+ cells in all observed TLSs (FIG. 1C, FIG. 5B), accounting for approximately 2% of all immune cells within TLSs (FIG. 1C). These results identified IL33 as a novel candidate signal that induced TLSs in human tumors.

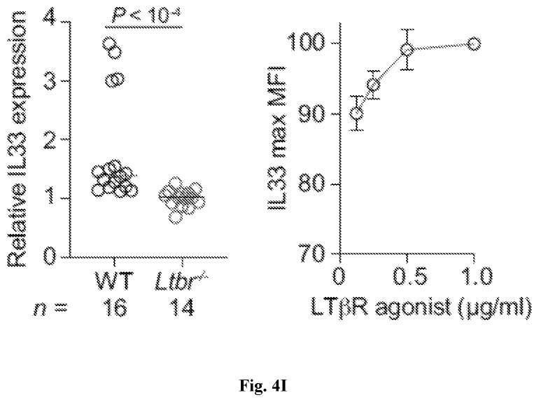

To investigate if the correlation between IL33 and TLSs in tumors was causative, we posited that if IL33 induced TLSs, this would likely be mediated by IL33's domain that is released extracellularly to coordinate its alarmin function by binding the receptor ST2(16). To test this, we administered IL33's alarmin domain (i.e., amino acids 112-270 oof IL33, referred to here as recombinant IL33 or “rIL33”) to Kras and p53-driven orthotopic PDAC mouse models (PDAC mice) with varying intratumoral T cell frequencies (FIG. 6)(17) to model the variable T cell density observed in human PDAC(18). Consistently, we found that rIL33 induced de novo TLSs in all five PDAC mouse models tested (FIG. 1D). Thus, these data suggested that IL33 is a novel alarmin that induces TLSs in cancer.

IL33 activates inflammatory ILC2s to express lymphotoxin in tumors

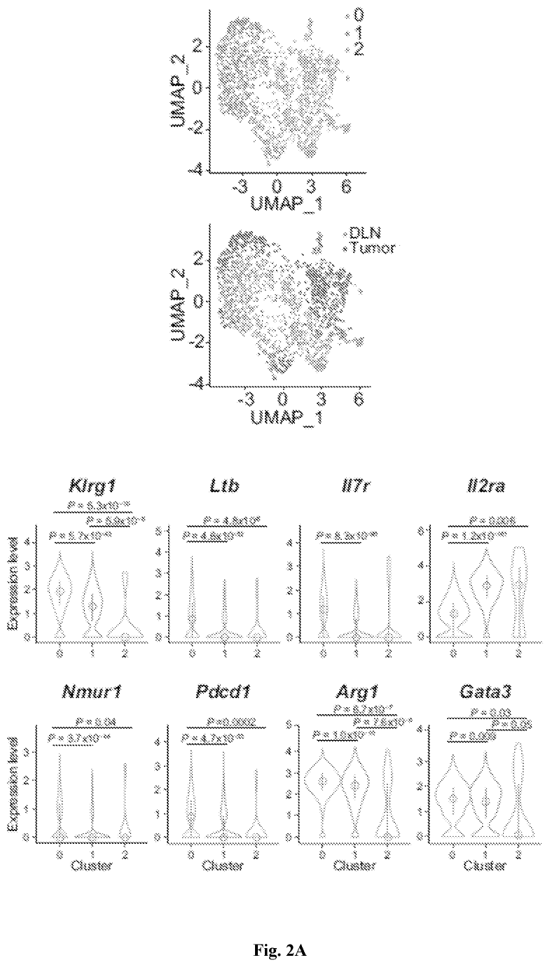

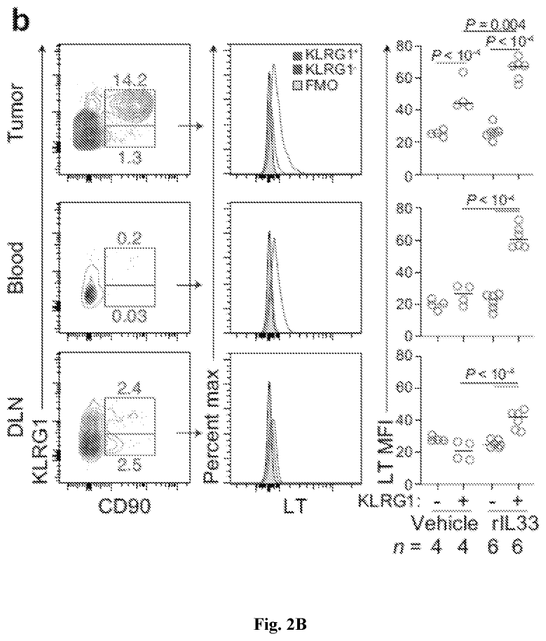

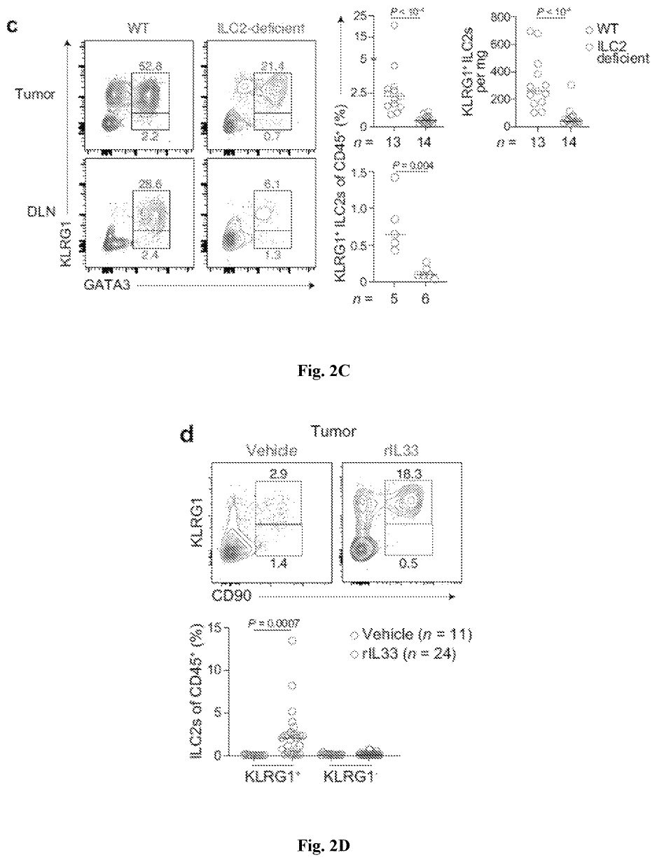

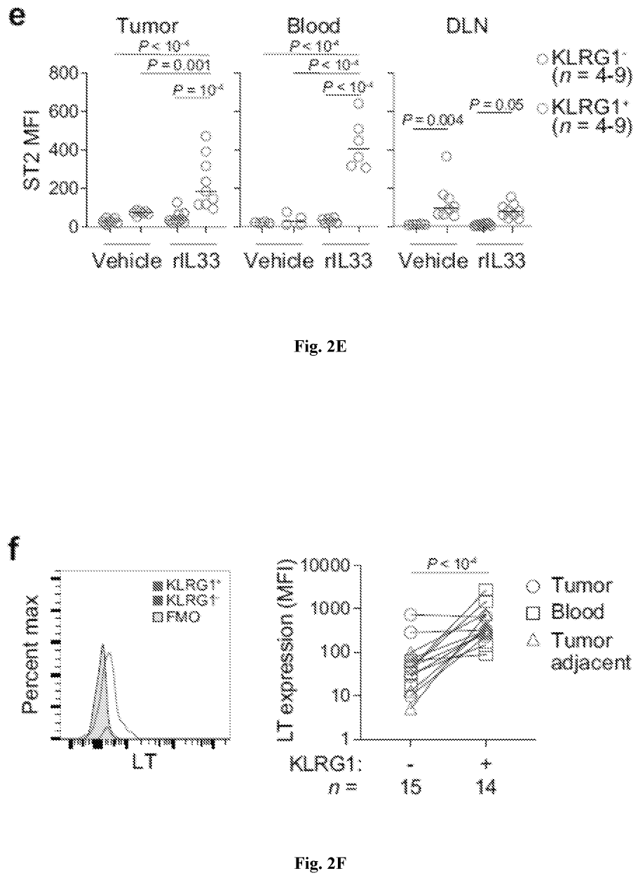

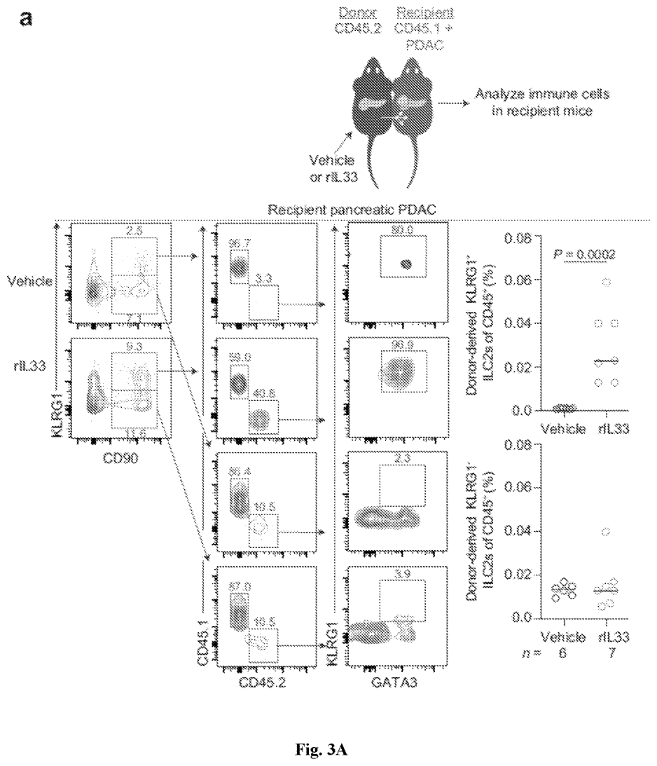

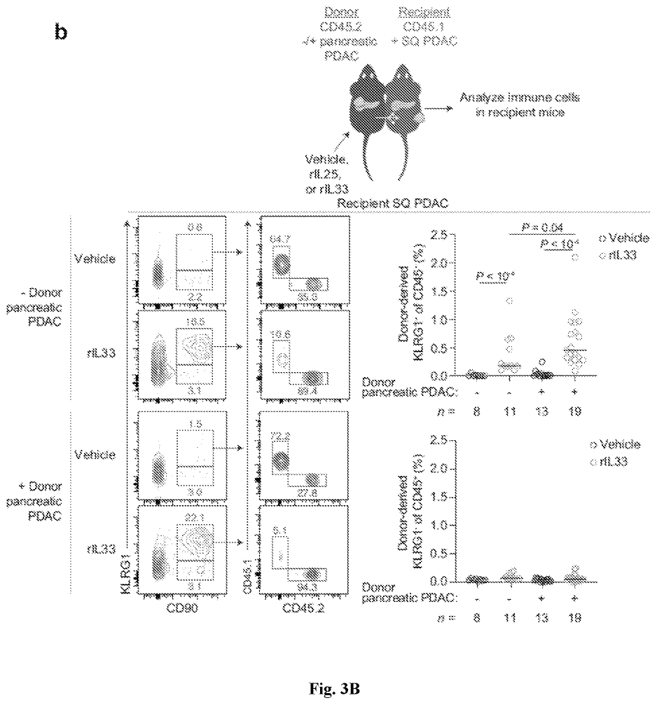

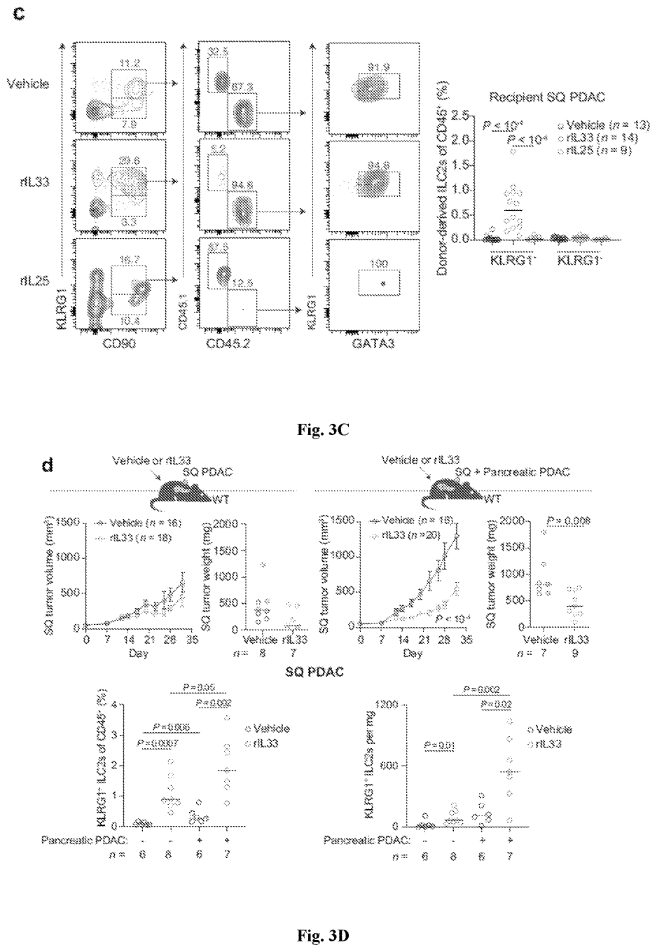

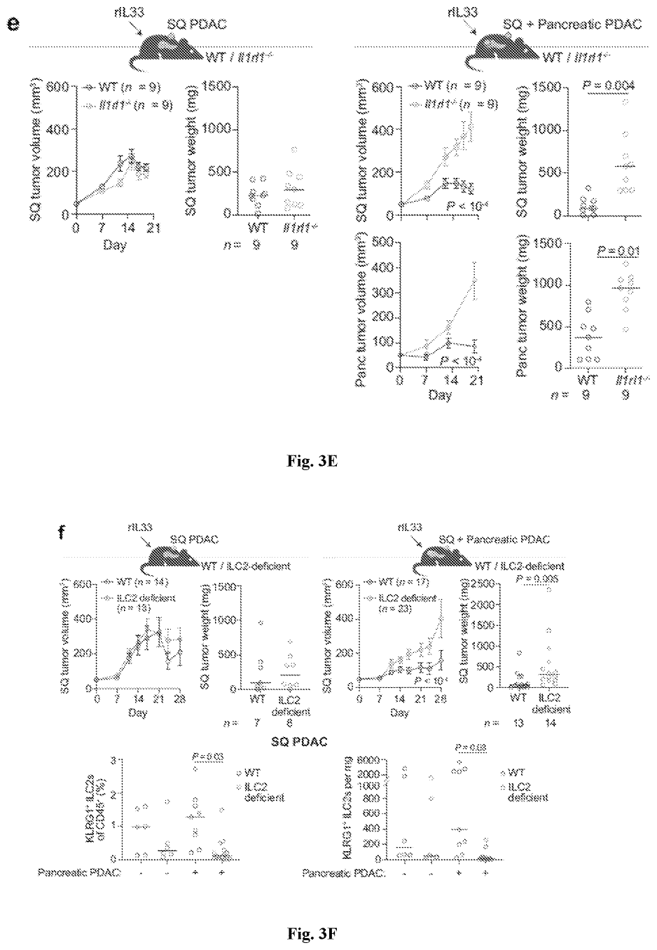

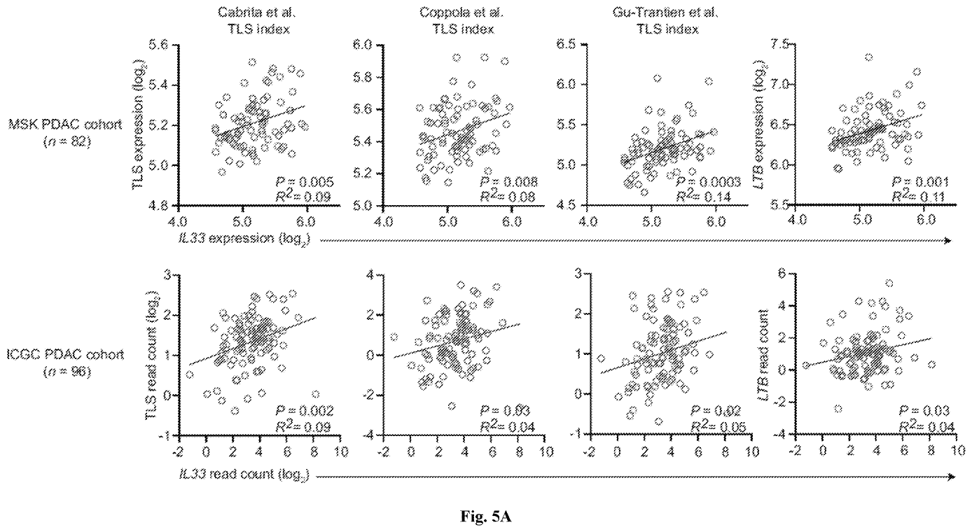

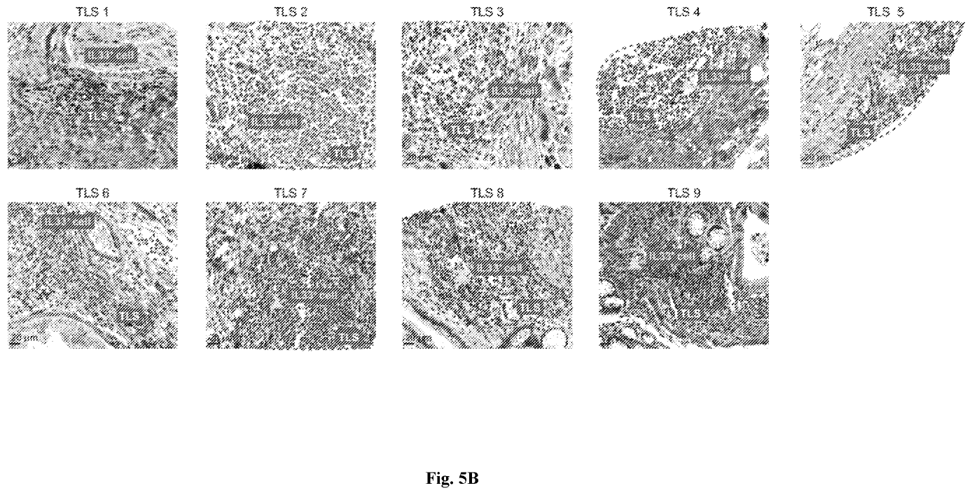

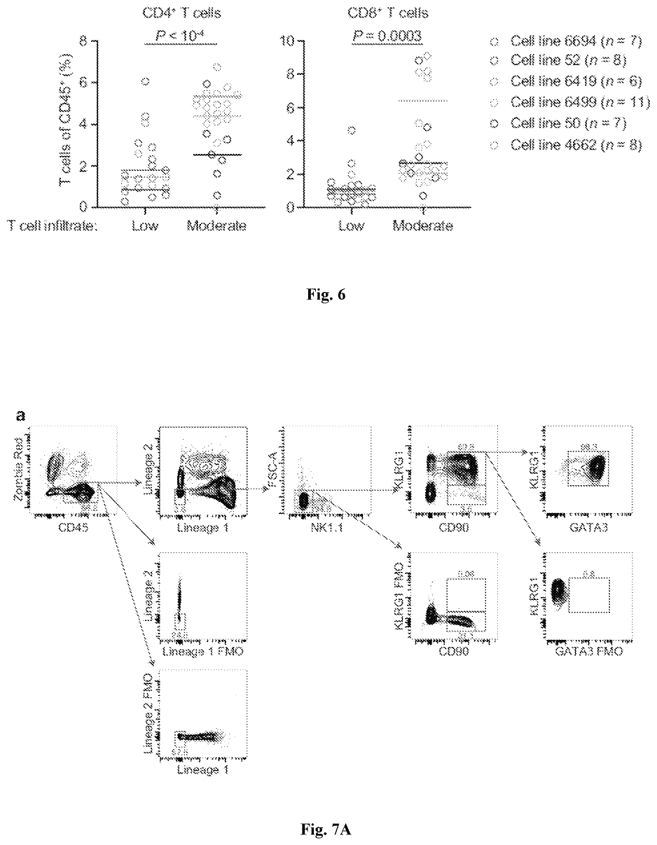

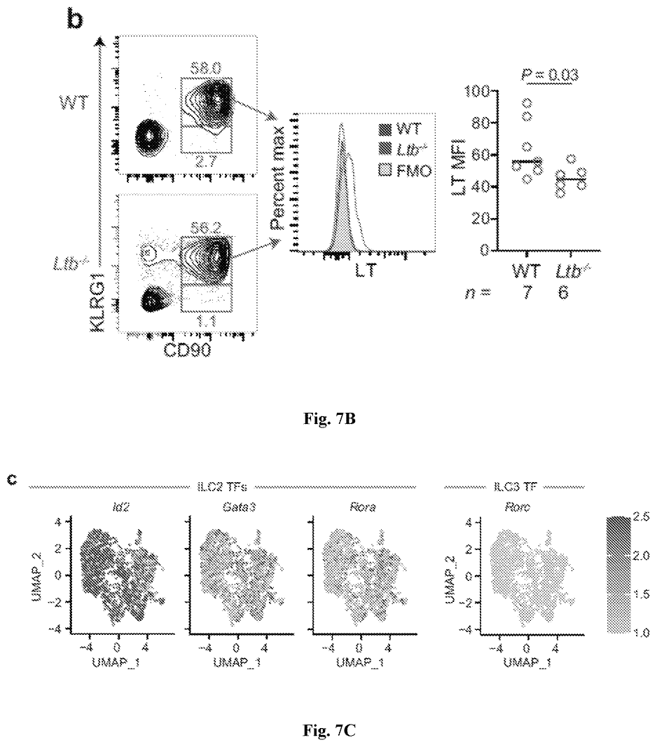

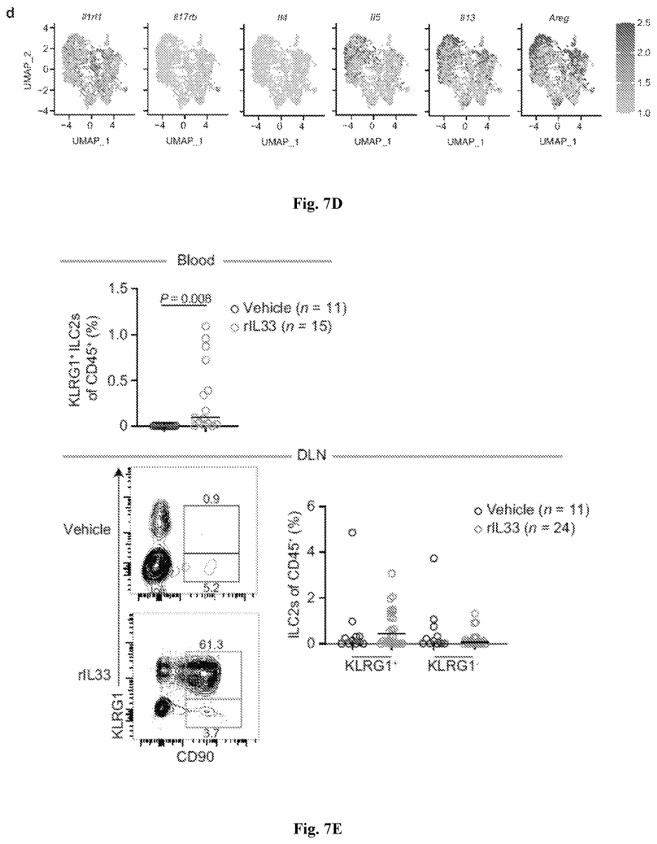

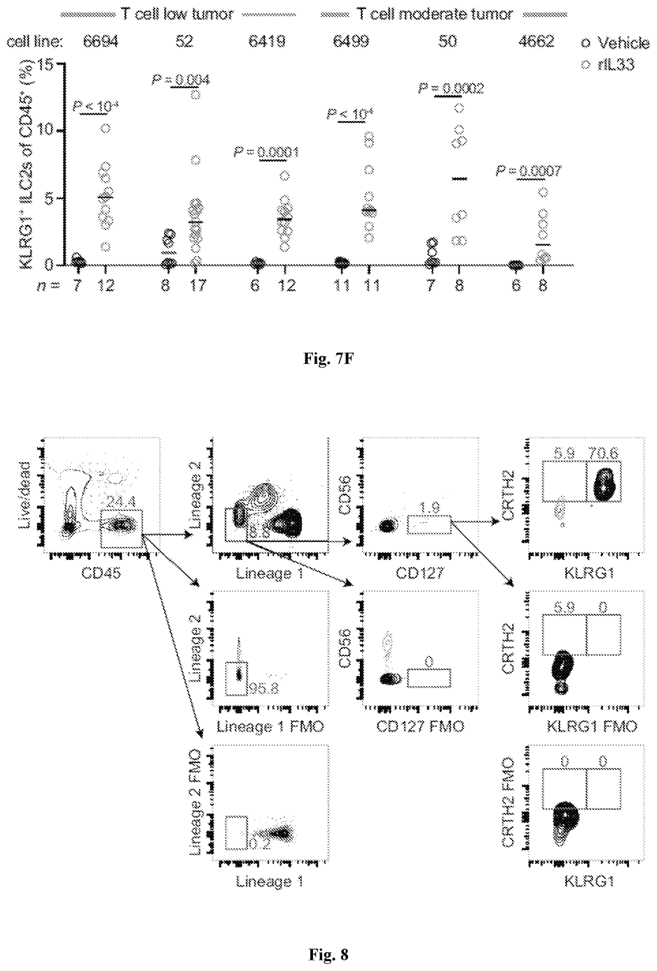

We next sought to identify the cells activated by rIL33 to induce TLSs. As we previously detected ST2+ ILC2s as rIL33's dominant cellular target in PDAC (19), we examined the single-cell transcriptomes of ST2+ ILC2s purified from tumors and tumor-draining lymph nodes (DLNs) from rIL33-treated PDAC mice (FIG. 2A), as previously described (19). We detected a dominant ILC2 population in tumors and DLNs that highly co-expressed the lymphocyte activation marker KLRG1 and genes essential for lymphoid organo-1(4,20,21) and neo-(3,22) genesis—Ltb and Il7r (cluster 0 in FIG. 2A; Extended Data FIG. 3a). KLRG1+ ILC2s (FIG. 7B) notably both expressed, and inducibly upregulated LTa1b2 (lymphotoxin [LT]) (FIG. 2B), the heterotrimeric cytokine that induces lymphoid tissue, to functionally resemble canonical lymphoid tissue inducer ILCs (LTi cells) that induce SLOs (23). However, unlike LTi cells and LTi-like ILC3s that developmentally require the transcription factor RORgt24, these putative lymphoid tissue-inducing KLRG1+ ILC2s derived distinctly from an ILC2-lineage—they expressed canonical ILC2 markers (FIGS. 7C & D) and were depleted in tumors and DLNs with lineage-specific deletion of the ILC2-transcription factor Rora25 (ILC2-deficient) (FIG. 2C). KLRG1+ ILC2s in tumors and DLNs also interestingly overexpressed genes (FIG. 2A, right—Klrg1, Nmur1, Pdcd1, Arg1, Gata3)2 (6-29) characteristic of inflammatory ILC2s (iILC2s) that migrate hematogenously to acutely infected tissues (26,27). Consistent with potential migratory capabilities, rIL33 expanded KLRG1+ ILC2s both in the blood and DLNs (FIG. 3E), and in a panel of PDACs from six mouse models (FIG. 2D, FIG. 3F), and furthermore, induced KLRG1+ ILC2s to upregulate ST226 (FIG. 2E). Importantly, we also detected rare KLRG1+ TLC2s infiltrating multiple human tumors that, like KLRG1+ TLC2s in mice, overexpressed LT (FIG. 2F, FIG. 8). In summary, as rIL33 activated ILC2s with putative migratory capacity to express LT, we hypothesized that IL33 may induce TLC2s to migrate to tumors and utilize the LT pathway to induce TLS.

### Inflammatory TLC2s Migrate to Control Tumors

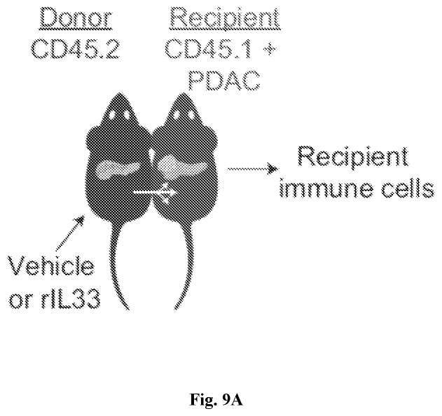

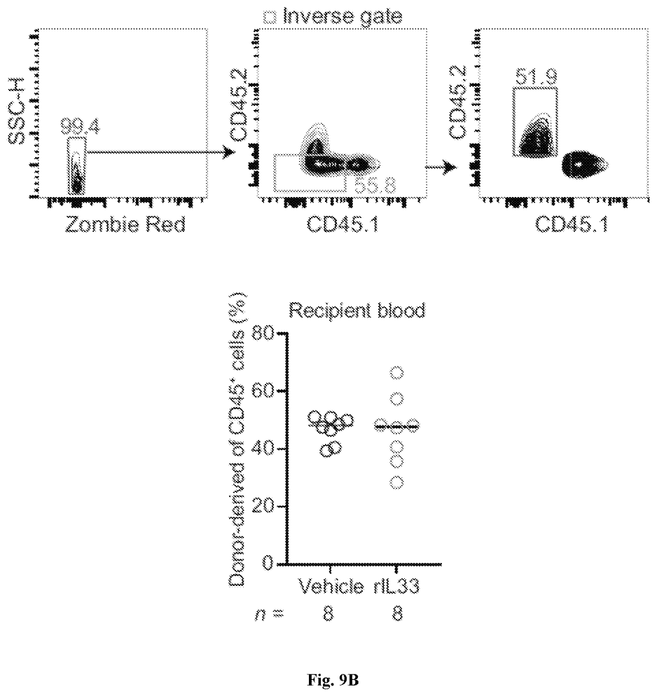

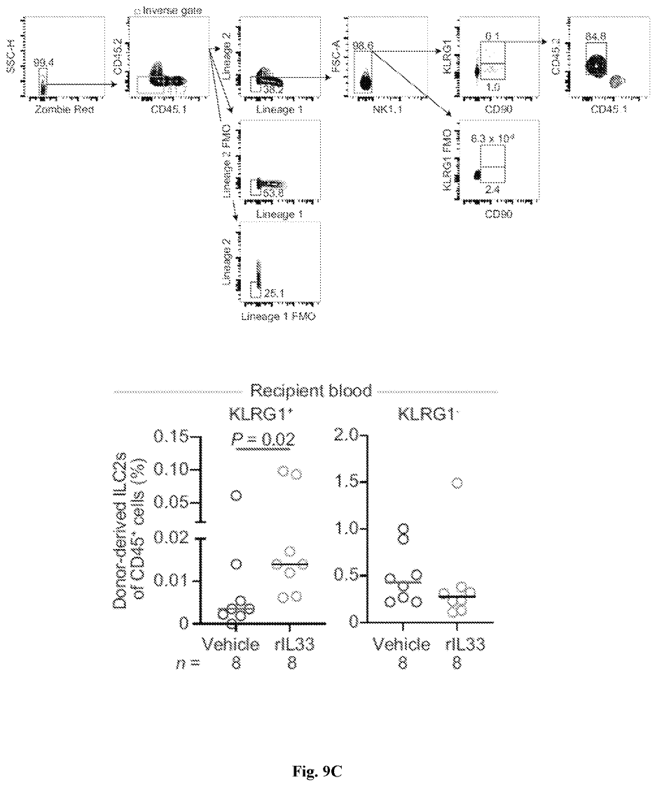

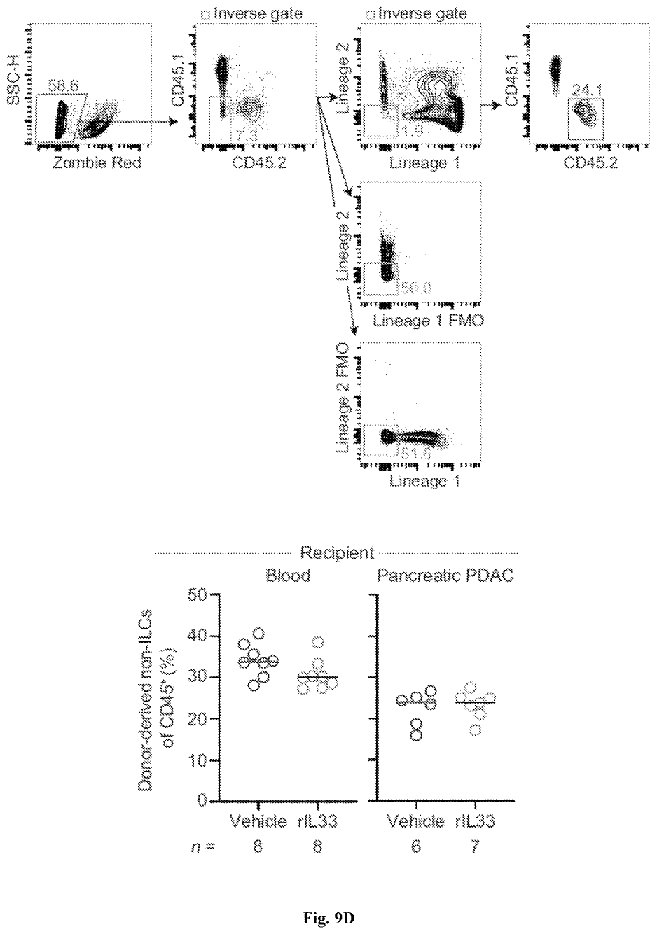

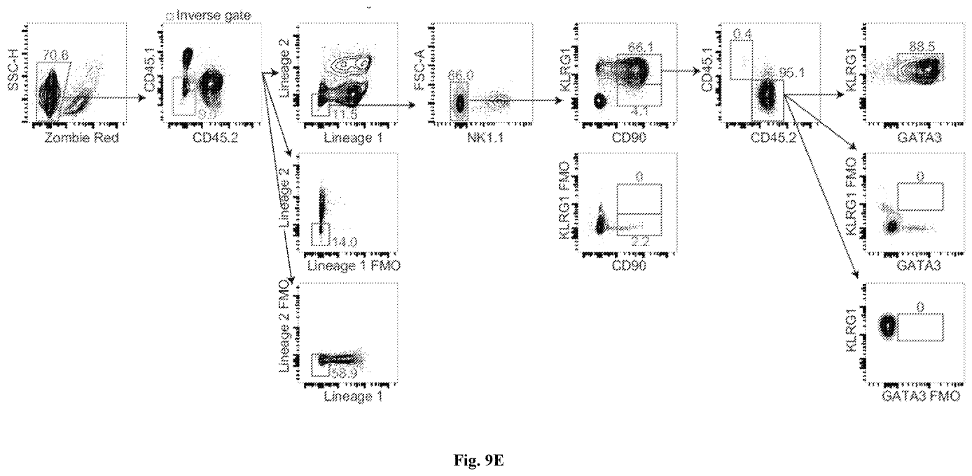

To test this hypothesis, we used parabiotic mice to investigate if, contrary to current presumption (19), KLRG1+ ILC2s in fact migrated hematogenously to tumors rather than arising from local tissue sources. We surgically connected congenic CD45.2 donor mice to CD45.1 PDAC recipients, administered rIL33 to donors, and searched for donor-derived ILC2s in recipient blood and tumors (FIG. 3A). We found donor and recipient-derived CD45+ immune cells in ˜1:1 ratios in recipient blood (FIGS. 9A & B). However, rIL33 selectively expanded donor-derived KLRG1+, but not KLRG1− ILC2s or non-ILC immune cells in recipient blood (FIGS. 9C & D) and tumors (FIG. 3A, FIGS. 9D & E). Thus, unlike currently presumed, KLRG1+ ILC2s in tumors contain bona fide migratory iILC2s that derive from hematogenous sources.

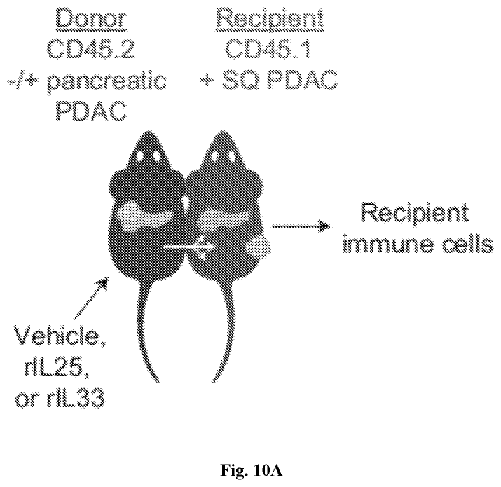

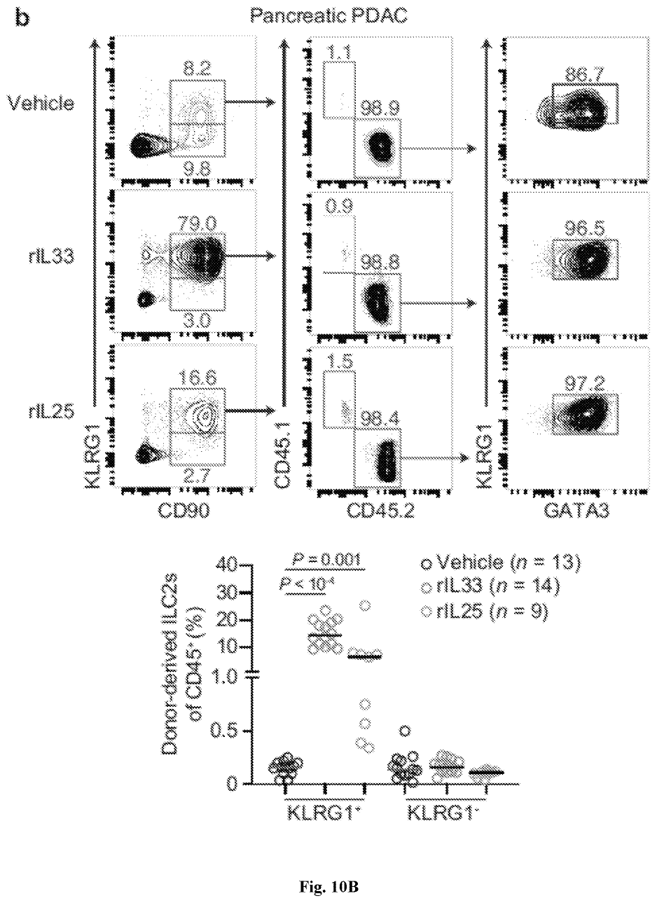

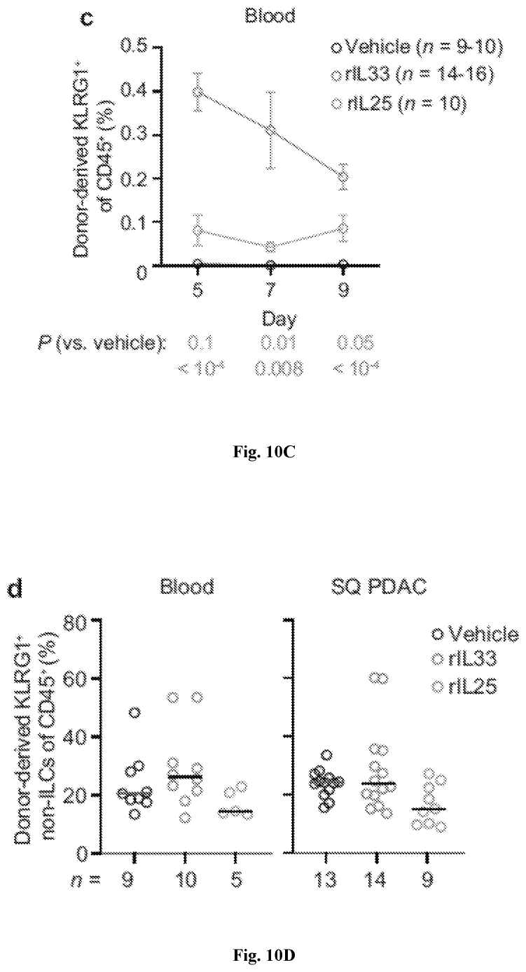

Recent evidence indicates acute local tissue injury extrudes iILC2s systemically to distant tissues(30), as an adaptation to both pair and toggle systemic tissue protection to fluctuating levels of local injury. We postulated that iILC2s would likely utilize similar migratory abilities to boost immunity in chronically inflamed “distant tumors”—namely, that local tumors would amplify iILC2 migration to distant tumors. To test this hypothesis, we established parabiotic mice, implanted PDACs in pancreata of donor mice to model local tumors, and in the skin (subcutaneous [SQ]) of recipient mice to model distant tumors. We next treated donor PDAC mice with rIL33 and measured donor-derived iILC2s in recipient blood (FIG. 10) and SQ PDACs (FIG. 3B). Donor iILC2s not only migrated to recipient pancreatic PDACs (FIG. 3A), but also to recipient SQ PDACs (FIG. 3B), to indicate iILC2s could migrate to tumors in diverse tissues. Interestingly, the presence of a pancreatic PDAC in donors increased rIL33-induced iILC2 migration to recipient SQ PDACs (FIG. 3B). This iILC2 migratory behavior to distant tumors required distinct cytokines from those involved in acute infections, as although both rIL3331 and rIL2526,27 expanded iILC2s in donor PDACs (FIGS. 10A&B), only rIL33 and not rIL2523 disseminated iILC2s from donors into recipient blood (FIG. 10C) and SQ PDACs (FIG. 3C). Neither rIL33 nor rIL25 induced non-ILC immune cells to migrate from donors to recipients (FIG. 10D), to indicate iILC2 migration to tumors was not consequent to migration of other immune cells. Thus, a local tumor stimulates iILC2s to migrate to a distant tumor. These results demonstrated that iILC2s migrate not only to acutely inflamed, but also to chronically inflamed tissues, and thus possess a conserved function to migrate to sites of tissue injury.

We next investigated if iILC2s migrated to distant tumors to boost immunity. We implanted mice with pancreatic (local) and/or SQ (distant) PDACs, treated these mice with rIL33, and examined SQ PDAC growth (FIG. 3D). In SQ PDAC mice, rIL33 minimally expanded intratumoral iILC2s densities, and failed to significantly restrict SQ PDAC growth (FIG. 3D, left), consistent with prior results(19). However, in dual PDAC mice, similar to ILC2 expansion in parabiotic mice (FIG. 3B), rIL33 expanded intratumoral iILC2s and restricted SQ PDAC growth (FIG. 3D, right). Notably, rIL33 did not control SQ PDAC growth in either Il1rl1−/− (FIG. 3E) or ILC2-deficient (FIG. 3F) dual PDAC mice, to indicate rIL33 required intact signaling through its receptor Il1rl1 (ST2) and ILC2s to control distant tumors. Therefore, these data indicated iILC2s migrate to distant tumors to restrict tumor growth.

### Inflammatory ILC2s Utilize Lymphotoxin to Induce Tertiary Lymphoid Structures in Tumors

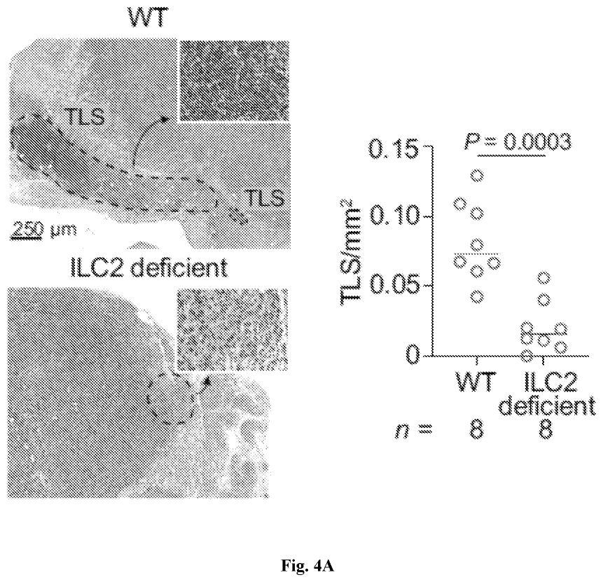

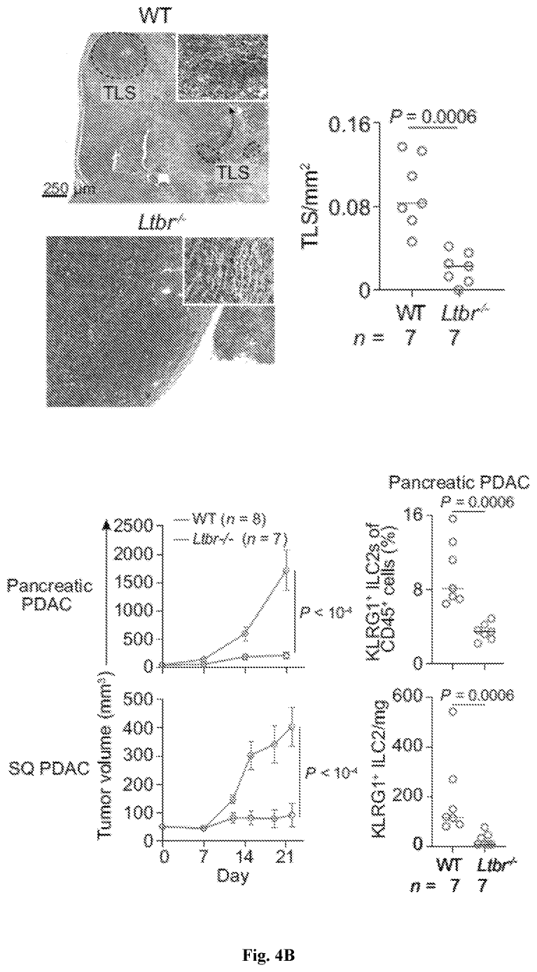

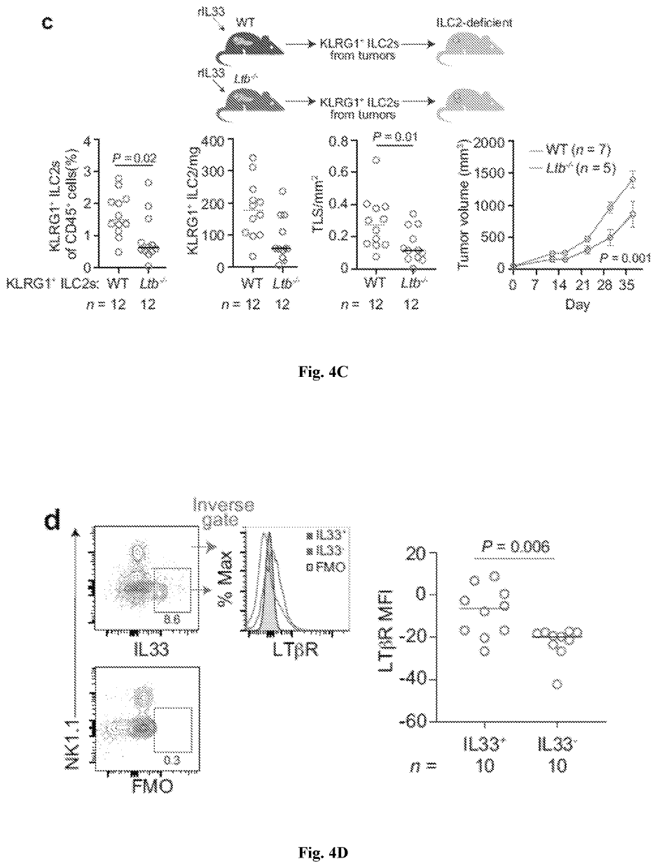

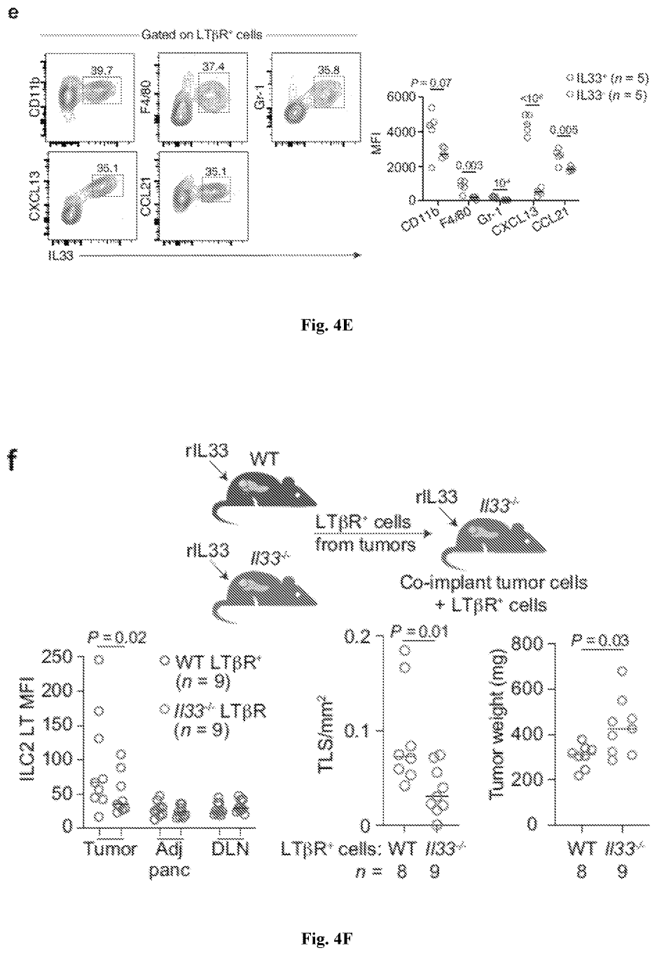

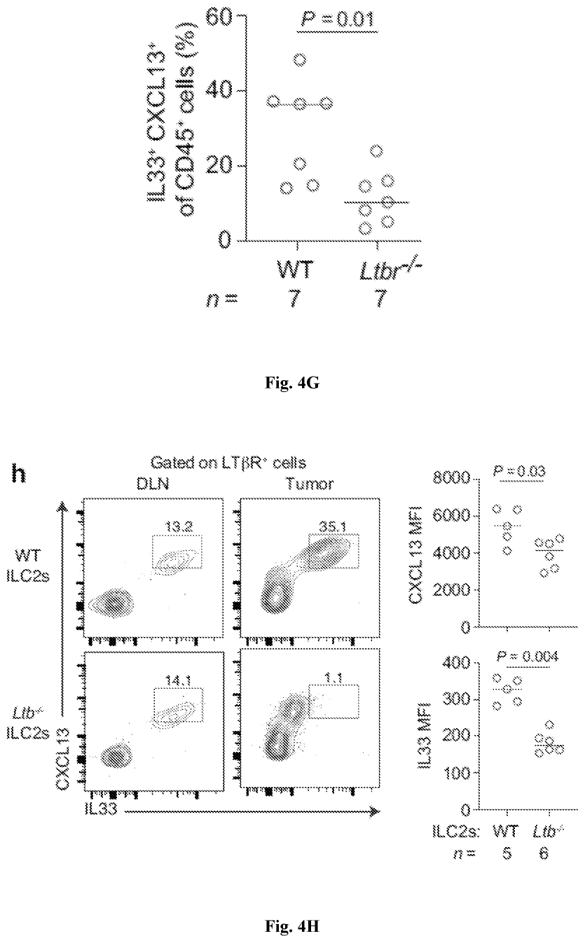

As rIL33 induced iILC2s to expand (FIG. 3A, B) and upregulate LT (FIG. 2B) in tumors, we reasoned iILC2s may serve as inducer cells to initiate TLSs and suppress tumors. To test this, we administered rIL33 to dual PDAC mice deficient in either ILC2s or LTbR, the obligate receptor of the LT pathway that mediates lymphoid tissue development. (32) rIL33 failed to induce TLSs in pancreatic PDACs in both ILC2-deficient (FIG. 4A) and Ltbr−/− dual PDAC mice (FIG. 4B, left), and also did not restrict either primary or distant tumors in these mice (FIG. 3F, FIG. 4B middle). Thus, rIL33 required ILC2s and a functional LT pathway to induce TLS and control tumors.

To explore if iILC2s utilized LT to directly induce TLSs, we examined iILC2 frequencies in rIL33-treated Ltbr−/− tumors to assess if iILC2 frequencies changed when functional LT signaling was absent. Interestingly, iILC2s were significantly reduced in tumors of Ltbr−/− mice (FIG. 4B, right), to suggest iILC2s utilize the LT-LTbR pathway to accumulate in tumors to possibly induce TLSs. To test this, we transferred iILC2s isolated from PDACs in wild-type (WT) or Ltb−/− mice into ILC2-deficient PDAC recipients (FIG. 4C, FIG. 11A). Consistently, Ltb deficiency impaired iILC2 ability to accumulate within tumors, induce TLSs, and control tumor growth (FIG. 4C). Collectively, these results demonstrated iILC2s utilize LT to accumulate in tumors, induce TLSs, and restrict growth.

In SLOs and TLSs, LT on inducer cells binds LTbR on stromal organizer cells to stimulate LT-inducing ligand expression, to thereby coordinate lymphoid neogenesis through a feed-forward loop.(33) As iILC2s seemingly functioned as inducer cells in a novel TLS pathway, we searched for the partner LTbR+ organizer cell. We curiously found in PDAC that IL33+ immune cells most highly express LTbR (FIG. 4D). These LTbR+IL33+ cells expressed markers of bone marrow-derived myeloid cells34 (FIG. 4E), and importantly, also CXCL13 and CCL21, the canonical lymphoid tissue organizing cytokines35 (FIG. 4E). Thus, as we also detected similar IL33+ immune cells within TLSs in PDAC patients (FIG. 1C), we posited that these IL33+ LTbR+ myeloid cells are candidate TLS organizer cells that functionally boost local IL33 production to amplify iILC2s and TLSs in tumors.

To test if LTbR+ myeloid cells functioned as TLS organizer cells, we first investigated if myeloid cell-derived IL33 regulated the ability of iILC2s to induce LT or TLSs in tumors. We implanted LTbR+ myeloid cells either proficient or deficient in IL33 into tumors of Il33−/− PDAC mice, and examined iILC2s and TLSs (FIG. 4F, FIG. 11B). Interestingly, LTbR+ myeloid cell-derived IL33 was sufficient to induce iILC2s in tumors to upregulate LT, induce TLS, and control PDACs (FIG. 4F), to indicate LTbR+IL33+ cells may function as TLS organizer cells in tumors. Indeed, like iILC2s, LTbR+IL33+ cells utilized the LT-LTbR pathway to persist in tumors, as we detected fewer IL33+ CXCL13+ cells in Ltbr−/− PDACs (FIG. 4G), and thus like iILC2s (FIG. 4B), required functional LT signaling to accumulate in tumors. To test if iILC2-derived LT bound LTbR on IL33+ cells to produce CXCL13 in tumors, we transferred WT or Ltb−/− iILC2s to ILC2-deficient PDAC recipients. iILC2 cell-intrinsic LT was sufficient to stimulate IL33+ LTbR+ cells in PDACs to significantly increase CXCL13 per-cell production, and to a lesser extent, increase CXCL13+ cell frequency, in tumors (FIG. 4H). To probe if LTbR signaling induced IL33 expression in myeloid cells, we examined IL33 expression in WT and Ltbr−/− myeloid cells to find LTbR-deficiency decreased IL33 expression (FIG. 4I, left). Consistently, stimulating LTbR on myeloid cells with an agonistic LTbR-Ig in vitro induced myeloid cells to upregulate IL33 in a dose-dependent manner (FIG. 4I, right). Together, these data indicated iILC2s and myeloid cells utilize the LT-LTbR pathway to reciprocally sustain their cell frequencies, produce CXCL13, and induce TLSs in tumors. Consequently, we identify iILC2s and myeloid cells as novel TLS inducer and organizer cells in tumors.

### The IL33-TLS Pathway can be Engineered for Cancer Immunotherapy

As we found that IL33 activates ST2+ iILC2s (FIG. 2D) to induce TLSs (FIG. 4A, C), and control tumors in an ST2-dependent manner (FIG. 3E), we reasoned that the IL33-ST2 pathway could be manipulated for cancer immunotherapy. We synthesized the human equivalent of mouse rIL33 (H-rIL33),

### Effects in Chronic Inflammatory Conditions

We performed experiments to determine if L33 generate TLSs in mouse models of both chronic inflammation and cancer.

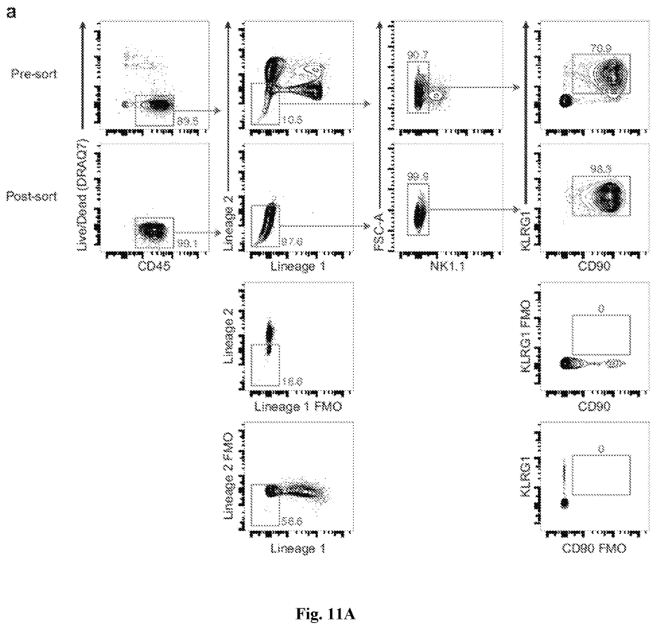

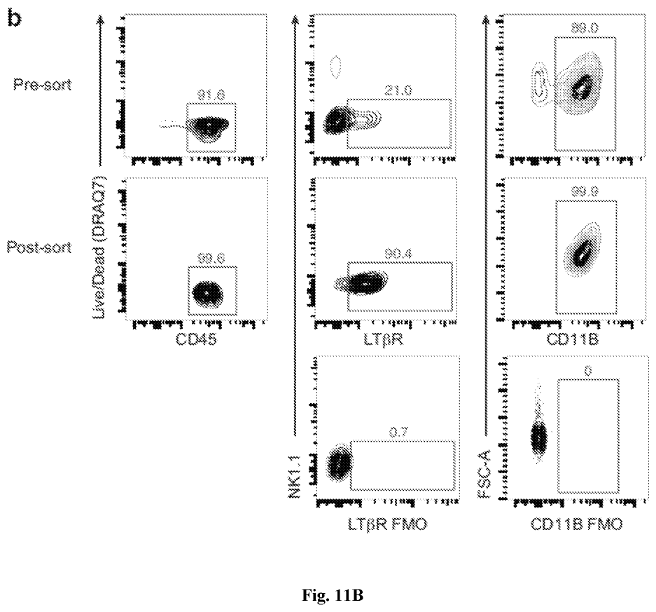

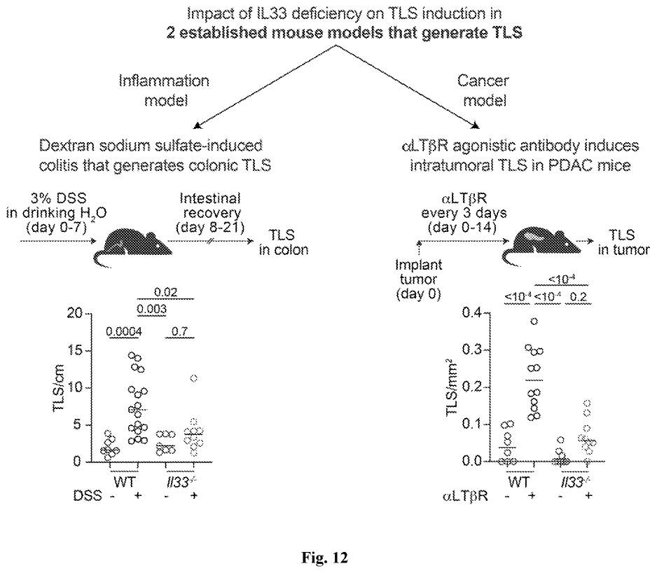

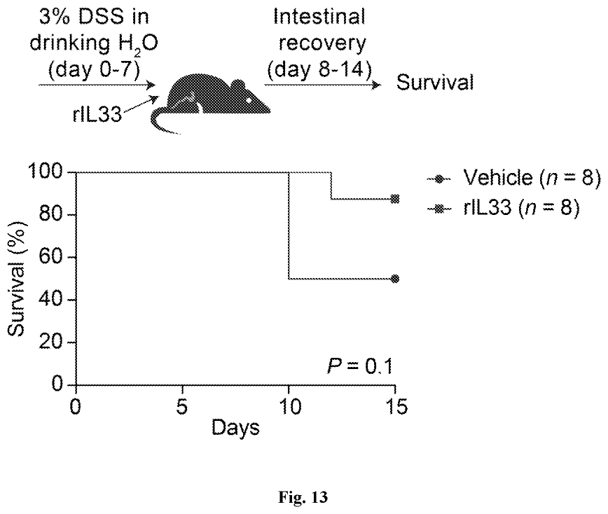

In a mouse model of dextran sulfate sodium (DSS)-induced inflammatory colitis that induces colonic TLSs, IL33−/− mice had significantly fewer colonic TLSs (P=0.009) and worsened survival compared to DSS-treated WT mice (P=0.0001). FIG. 12 provides a schematic of the DSS colitis model of inflammation-induced colonic tertiary lymphoid structures (TLS). Briefly, wild-type (WT) or IL33−/− mice were treated with 3% DSS in drinking water for 7 days and allowed to recover for 14 days (no DSS exposure). TLS in the colon were quantified. Treatment of mice with rIL33 in this model lead to improved survival. FIG. 13 provides results from a study in which wild-type (WT) mice were treated with 3% dextran sodium sulfate (DSS) in drinking water and rIL33 (500 ng) daily for 7 days, after which they were allowed to recover for 7 days (no DSS exposure; rIL33 3 times/week), and survival was assessed (bottom).

Thus, IL33 induces TLS formation in both chronic inflammatory conditions and cancer, leading to improved outcomes.

### DISCUSSION

Our results uncover a new pathway through which a danger signal activates a migratory cellular lymphogenic response in tissues. The identification of a novel TLS pathway has implications for both cancer immunotherapy and chronic inflammatory diseases. This previously unknown function for the IL33-ST2 pathway to induce TLSs notwithstanding, we also identify ILC2s and myeloid cells as novel inducer and organizer cells that generate TLSs, and thereby uncover an unrecognized mechanism of action through which ILC2s control tumors. In sum, we discover new molecules and cells that induce lymphoneogenesis in chronically inflamed tissues.

### Methods

C57BL/6 (wild-type, WT, CD45.2) and C57BL/6 CD45.1 mice were purchased from Jackson Laboratory. For all experiments, 6-12-week-old mice were matched by age and sex and randomly assigned to specific treatment groups, with at least two independent experiments performed throughout. Sample sizes for experiments were determined without formal power calculations. Animals were bred and maintained in a specific pathogen-free animal facility, and all experiments were conducted in accordance with an Institutional Animal Care and Use Committee (IACUC) approved protocol at Memorial Sloan Kettering Cancer Center (MSKCC) and in compliance with all relevant ethical regulations.

### Cell Lines and Animal Procedures

All tumor cell lines were derived from KPC (Pdx1-Cre; LSL-KrasG12D/+; LSL-Trp53R172H/+) or KPCY (Pdx1-Cre; LSL-KrasG12D/+; LSL-Trp53R172H/+; Rosa26YFP/YFP). All cell lines were authenticated as bona fide PDAC cell lines based on histopathologic verification by a dedicated pancreatic cancer pathologist. HEK-Blue-IL33 cell line (Invivogen) was cultured in DMEM (Gibco), 10% FBS (Gibco), penicillin (100 IU/ml), streptomycin (100 μg/ml), and 100 μg/ml Normocin (Invivogen) at 37° C. in 5% CO2. All other cell lines were cultured in DMEM with 10% FBS and glutamine (2 mM) at 37° C. in 5% CO2. All cell lines were regularly tested using MycoAlert Mycoplasma Detection Kit (Lonza).

Tumors were established orthotopically (pancreatic, “PDAC” mice) or subcutaneously (SQ) as previously described(19). Briefly, for orthotopic implantation, mice were anesthetized using a ketamine/xylazine cocktail and a small (7 mm) left abdominal side incision was made. Tumor cells (10⁶ cells for KPC-4662; 10⁵ cells for all others) were suspended in Matrigel (Becton Dickinson), diluted 1:1 with cold phosphate-buffered saline (PBS) in a total volume of 50 ml, and injected into the tail of the pancreas using a 26-gauge needle. Successful injection was verified by the appearance of a fluid bubble without intraperitoneal leakage. The abdominal wall was closed with absorbable Vicryl RAPIDE sutures (Ethicon), and the skin was closed with wound clips (Roboz). For SQ implantation, tumor cells (5×10⁵ cells for KPC-4662; 5×10⁴ cells for all others) were resuspended in sterile PBS and implanted subcutaneously. All tumors were established with KPC-4662 unless otherwise specified. For orthotopic tumors, tumor volumes were measured using serial ultrasound (Vevo 2100 Linear Array Imaging and Vivo LAB Version 3.1.1, Fuji Film Visual Sonics) as previously described (25). Tumors were harvested at indicated time points. To assess T cell infiltrates in PDACs derived from different cell lines (FIG. 6), tumors were harvested at time points when they were of equivalent volumes. For SQ tumors, tumor length and width were measured every 2-3 days with calipers, and tumor volumes were calculated as Volume=½ Length×Width2. Mice were sacrificed at the indicated time points and processed for histology or flow cytometry. For survival analyses, survival was determined by a tumor volume of 3500 mm³ or mouse health requiring euthanasia as defined by institutional IACUC guidelines. No mouse tumors exceeded IACUC-defined maximal tumor volumes of 32 cm³. No blinding was performed in experimental mouse interventions, as knowledge of the treatment groups was required.

### Parabiosis

Six-week-old female congenic CD45.1 and CD45.2 mice were surgically connected as previously described (27). Briefly, mice of similar body weight were co-housed 2 weeks prior to surgery and treated with prophylactic antibiotics (Sulaftrim diet, WF Fisher and Son, Inc.) starting the day prior to surgery. Corresponding lateral skin incisions were made from elbow to knee on each mouse, fore- and hindlimbs were sutured together, and the skin incisions were closed. After surgery, mice were maintained on a diet supplemented with prophylactic sulfamethoxazole for 2 weeks, followed by a normal diet thereafter. After confirming blood chimerism at 4 weeks following parabiotic surgery, pancreatic and/or SQ PDACs were implanted as described above. Peripheral blood was collected from the submandibular vein of recipient mice using a golden rod animal lancet (Medipoint, Inc.) at 5, 7, and 9 days after tumor implantation. Parabionts were euthanized and organs were harvested 14 days after tumor implantation.

### Recombinant H-rIL33

H-rIL33 proteins were generated at GenScript Biotech (Piscataway, NJ). Briefly, target DNA sequences were codon optimized, synthesized, and subcloned into a cytomegalovirus promoter-driven expression vector following the human IL2 signal peptide sequence. The proteins were expressed by transient transfection in HD CHO cells and purified by affinity chromatography, followed by size exclusion chromatography to obtain the desired purity.

The purified protein was analyzed by SDS-PAGE, Western blot, and HPLC analysis to determine the molecular weight and purity Recombinant IL33, IL25, and H-rIL33 treatment Following tumor implantation, mice were treated with intraperitoneal (i.p.) injections of 500 ng carrier-free recombinant murine IL3319, IL25 (R&D Systems), or recombinant human IL33 (H-rIL33, Proteos, Inc.) daily for 7 days, and then every 2 days thereafter.

### Human Samples

All tissues were collected at MSKCC following study protocol approval by the MSKCC Institutional Review Board. Informed consent was obtained for all patients. The study was performed in strict compliance with all institutional ethical regulations. All tumor samples were surgically resected primary PDACs (for tumor transcriptomic profiling), or surgically resected primary human PDAC or colorectal liver metastases (for flow cytometry). The human PDAC tissue microarrays used have been previously described (19).

Tumor transcriptomic profiling: Primary PDACs from surgically resected PDAC patients were randomly selected to undergo transcriptomic profiling as previously described(19). Briefly, total RNA from fresh frozen OCT-embedded tumors was extracted using TRIzol RNA Isolation Reagents (15596-026, Life Technologies), qualified on an Agilent BioAnalyzer, quantified by fluorometry (Ribogreen), and prepared for whole-transcriptome expression analysis using the WT Pico Reagent Kit (Affymetrix). RNA was then amplified using low-cycle PCR followed by linear amplification using T7 in vitro transcription technology. The cRNA was then converted to biotinylated sense-strand DNA hybridization targets, and hybridized to GeneChip Human Transcriptome Array 2.0 (Affymetrix), and scanned using the GeneChip Scanner 3000. Data were analyzed using R (version 4.0.3).

### Cell Isolation

Mouse and human PDACs were mechanically dissociated and incubated in collagenase (collagenase II for murine tumors, collagenase IV for human tumors, both 5 mg/ml; Worthington Biochemical Corp., Fisher Scientific), DNAse I (0.5 mg/ml; Roche Diagnostics), and Hank's balanced salt solution (Gibco, Fisher Scientific) for 30 minutes at 37° C. Digestion was then quenched with fetal bovine serum (FBS, Life Technologies). Digested tumors and DLNs were then mechanically disassociated and filtered through 100- and 40-mm nylon cell strainers (Falcon, Fisher Scientific) using PBS with 5% FBS (Life Technologies) and 2 mM EDTA (pH8.0, Invitrogen). Spleens were mechanically dissociated and filtered through 70- and 40-mm nylon cell strainers (Falcon, Fisher Scientific) using PBS with 5% FBS and 2 mM EDTA, followed by RBC lysis (RBC lysis buffer, Invitrogen Scientific). Peripheral blood was processed with RBC lysis and filtered through 40-mm nylon cell strainers. Mouse Fc receptors were blocked with FceRIII/II-specific antibody (1 μg per 1×10⁶ cells; clone 2.4G2, Bio XCell).

### ILC2 and Myeloid Cell Adoptive Transfer

CD45.1 C57Bl/6, Ltb−/−, or Il33−/− orthotopic PDAC mice were treated with 500 ng carrier-free recombinant murine IL33 (R&D Systems) in sterile PBS daily for 10 days. For ILC2 transfer, live, CD45+, lineage-, CD90+, KLRG1+ ILC2s from tumors were sort-purified to 98% purity at day 10 post-implantation using an Aria Cell Sorter (BD Biosciences) (FIG. 11A). 5×10⁵ ILC2s were immediately transferred to orthotopic PDAC tumor-bearing Il7rCre/+Rorafl/fl CD45.2 mice via i.p. injection 3 days after tumor implantation. For myeloid cell transfer, live, CD45+, NK1.1−, CD11b+, LTbR+ cells were sort-purified to 90% purity using an Aria Cell Sorter (Extended Data FIG. 7b). For adoptive transfer, 5×105 CD11b+, LTbR+ cells were mixed with tumor cell suspensions and injected into the tail of the pancreas using a 26-gauge needle. rIL33 treatment (500 ng per mouse as described above) was initiated in recipient mice on the day of ILC2 or myeloid cell transfer until the day of tissue harvest. Tissues were harvested at the indicated time points.

### Flow Cytometry

All samples were analyzed on a FACS LSR Fortessa (BD Biosciences). Mouse ILC2s were defined as live, CD45+, lineage− (CD3, CD5, NK1.1, CD11b, CD11c, CD19, FceR1), CD90+. All live, CD45+, lineage−, CD90+ cells were GATA3+(Extended Data FIG. 3a). Mouse iILC2s were defined as live, CD45+, lineage− (CD3, CD5, NK1.1, CD11b, CD11c, CD19, FeeRI), CD90+, KLRG1+. Human ILC2s were defined as live, CD45+, lineage-(CD3, CD5, CD56, CD11b, CD11c, CD14, CD16, CD19, TCRa/b, FeeRI), CD127+, CRTH2+. Human KLRG1+ ILC2s were defined as live, CD45+, lineage− (CD3, CD5, CD56, CD11b, CD11c, CD14, CD16, CD19, TCRa/b, FeeRI), CD127+, CRTH2+, KLRG1+. Murine cells were stained with the following antibodies: from Biolegend CD3 (clone 145-2C11, BV711), CD4 (clone RM4-5, BV711 and BV786), CD45 (clone 30-F11, Pacific Blue), CD45.1 (clone A20, BV711 and APC-Cy7), CD45.2 (clone 104, Pacific Blue), CD8 (clone 53-6.7, BV510), KLRG1 (clone 2F1/KLRG1, BV510), LTBR (clone 5G11, PE-Cy7), and Zombie Red Fixable Viability dye (Cat. #423110); from BD Biosciences, CD11b (clone M1/70, Alexa Fluor 700, APC, and APC-Cy7), CD5 (clone 53-7.3, APC), CD11c (clone HL3, APC), CD90.2 (clone 53-2.1, BV786), Gata3 (clone L50-823, BV711 and PE), Gr-1 (clone RB6-8C5, BV605), NK1.1 (clone PK136, BV650 and APC), DRAQ7 (Cat. #51-9011172); from Invitrogen Scientific CD19 (clone eBio1D3, Alexa Fluor 700), CD3 (clone 17A2, Alexa Fluor 700), CD8 (clone 53-6.7, Alexa Fluor 700), CXCL13 (clone DS8CX13, APC), F4/80 (clone BM8, PE-Cy5), FceR1 (clone MAR-1, APC), IL33 (clone 396118, PE), and TCRVb (clone MR-9-4, Alexa Fluor 700).

To detect mouse LT expression, single cell suspensions were incubated with recombinant mouse LTbR-Fc chimeric protein (1 μg/ml, R&D systems) in PBS with 5% fetal bovine serum (FBS) and 4 mM EDTA for 30 minutes in the dark at 4° C., then followed by incubation with a secondary antibody (goat anti-mouse IgG2a conjugated, Invitrogen) for 30 minutes in the dark at 4° C. For CCL21 detection, fixed and permeabilized single cell suspensions were incubated with anti-CCL21 antibody (Cat. #PA5-47016, Invitrogen) in Brilliant Stain Buffer (BD Bioscience) for 30 minutes in the dark at 4° C. followed by incubation with a secondary antibody (rabbit anti-goat IgG, Invitrogen) for 30 minutes in the dark at 4° C.

Human cells were stained with the following antibodies: from BD Biosciences, GATA3 (clone L50-823, PE); from Biolegend, CD11b (clone ICRF44, APC), CD45 (clone HI30, Pacific Blue), CD56 (clone HCD56, BV605), CRTH2 (clone BM16, PerCP/Cy5.5 and PE), FceR1 (clone AER-37, APC), KLRG1 (clone 2F1/KLRG1, BV510), TBET (clone 4B10, BV711), and TCRa/b (clone IP26, APC); from Invitrogen Scientific, CD14 (clone 61D3, APC), CD16 (clone CB16, APC), CD11c (clone 3.9, APC), CD127 (clone RDR5, FITC), CD3 (clone OKT3, Alexa Fluor 700), CD5 (clone L17F12, APC), and CD19 (clone HIB19, AF700).

To detect human LT expression, single cell suspensions were incubated with recombinant human LTbR-Fc chimeric protein (2 μg/ml, R&D systems, 629-LR) in PBS with 1% FBS and 0.5 mM EDTA (pH8.0, Invitrogen) for 30 minutes in the dark at 4° C. followed by incubation with a secondary antibody (mouse anti-human IgG, Invitrogen) for 30 minutes in the dark at 4° C. All samples for flow cytometry were from prospectively collected unselected patients.

### Hematoxylin and Eosin Staining

Pancreatic tumors were cut into 2-mm-thick slices and fixed in 4% paraformaldehyde solution (Electron Microscopy Sciences, Inc.), embedded in paraffin, stained with hematoxylin and eosin, and scanned on the Panoramic Scanner (3DHistech, Budapest, Hungary) with the 20×/0.8NA objective. The number of TLSs were determined in at least 3 sections using QuPath (ver.0.2.3; https://qupath.github.io/). A compact aggregate of lymphocytes >5,000 μm2 was considered as a TLS47.

### Immunohistochemistry

Immunohistochemistry was performed on previously described human PDAC tissue microarrays(19). Briefly, paraffin embedded tissue sections were deparaffinized with EZPrep buffer (Ventana Medical Systems). Antigen retrieval was performed with CC1 buffer (Ventana Medical Systems), followed with Background Buster solution (Innovex). Avidin-biotin blocking solution (Ventana Medical Systems) was then used to block tissue sections for 30 minutes. Sections were incubated with anti-human IL33 antibody (AF3625, R&D System) for 4 hours, followed by 60 minutes with biotinylated rabbit anti-goat IgG (Vector labs) at 1:200 dilution. IL33 positivity was detected with a DAB detection kit (Ventana Medical Systems). Any cell demonstrating cytoplasmic or nuclear IL33 positivity was designated to have positive staining. Nucleated cells in a TLS were determined and counted using the Analyze Particles function in ImageJ (ver. 2.3.0, NHI, USA). IL33+ cells in a TLS were counted manually.

### Immunofluorescence

Paraffin embedded tissues were sliced into 7-μm sections. Multiplex immunofluorescent staining was performed using a Discovery XT processor (Ventana Medical Systems) as described29.

B220: First, sections were incubated with anti-B220 (clone RA3-6B2, BD Biosciences) for 6 hours, followed by 60 minutes incubation with biotinylated horse anti-goat IgG (Vector Laboratories) at 1:200 dilution. Detection was performed with Streptavidin-HRP D (Ventana Medical Systems), followed by incubation with Tyramide Alexa Fluor 594 (Invitrogen) prepared according to the manufacturer's instructions with predetermined dilutions.

CD3: Next, sections were incubated with anti-CD3 (Cat. #A0452, DAKO) for 6 hours, followed by 60 minutes incubation with biotinylated goat anti-rabbit IgG (Vector Laboratories) at 1:200 dilution. Detection was performed with Streptavidin-HRP D (Ventana Medical Systems), followed by incubation with Tyramide Alexa 488 (Invitrogen) prepared according to the manufacturer's instructions with predetermined dilutions.

Lyve-1: Finally, sections were incubated with anti-Lyvie-1 (Cat. #AF2125, R&D systems) for 6 hours, followed by 60 minutes incubation with biotinylated goat anti-rabbit IgG (Vector Laboratories) at 1:200 dilution. Detection was performed with Streptavidin-HRP D (Ventana Medical Systems), followed by incubation with Tyramide Alexa 647 (Invitrogen) prepared according to the manufacturer's instructions with predetermined dilutions. After staining, slides were counterstained with DAPI (Sigma Aldrich) for 10 minutes and cover-slipped with Mowiol.

### Single-Cell RNA Sequencing

Library preparation, sequencing, and post-processing for single-cell immune profiling has been previously reported19. Briefly, ST2+ ILC2 cells were purified from pancreatic KPC tumors and DLNs from mice treated with rIL33 for 10 days. scRNA-seq libraries were prepared based on manufacturer's recommendations (Chromium Single Cell V(D)J User Guide PN-1000006, 10× Genomics). Cell suspensions (85-90% viable) at a concentration between 90 and 200 cells/l were loaded onto to the 10× Genomics Chromium platform to generate Gel Beads-inEmulsion (GEM), targeting about 2,000 single cells per sample. After GEM generation, the samples were incubated at 53° C. for 45 min in a C1000 Touch Thermal cycler with 96-Deep Well Reaction Module (BioRad) to generate polyA cDNA barcoded at the 5′ end by the addition of a template switch oligo (TSO) linked to a cell barcode and Unique Molecular Identifiers (UMIs). After breaking the GEMs, the single-strand cDNA was cleansed with DynaBeads MyOne Silane Beads (Thermo Fisher Scientific). The cDNA was then amplified for 16 cycles (98° C. for 45 s; 98° C. for 20 s, 67° C. for 30 s, 72° C. for 1 h), following which the cDNA quality was assessed using an Agilent Bioanalyzer 2100, obtaining a product of about 1,200 bp. cDNA (50 ng) was enzymatically fragmented, end repaired, A-tailed, subjected to a double-sided size selection with SPRI select beads (Beckman Coulter), and ligated to adaptors provided in the kit. Within each library, a unique sample kit was then introduced through 14 cycles of PCR amplification using the indexes provided in the kit (98° C. for 45 s; 98° C. for 20 s, 54° C. for 30 s, 72° C. for 20 s×14 cycles; 72° C. for 1 min; held at 4° C.). A second double-sided selection was then performed on the indexed libraries, following which libraries were quantified using Qubit fluorometric quantification (Thermo Fisher Scientific). An Agilent Bioanalyzer 2100 was used to assess the quality (average library size 450 bp), following which cDNA was amplified with 18 cycles, and sample index with 16 cycles. Diluted libraries were then clustered using a NovaSeq600 on a paired-end read flow cell, sequenced for 28 cycles on R1 (10× barcode and the UMIs), followed by 8 cycles of 17 index (sample index), and 89 bases on R2 transcript, obtaining approximately 100 million clusters per samples. Primary processing of sequencing images was done using Illumina's Real Time Analysis software (RTA). 10× Genomics Cell Ranger Single Cell Software suite v3.0.2 (https://support.10×genomics.com/single-cell-gene-expression/software/pipelines/latest/what-is-cellranger) was used to demultiplex samples, align to mouse genomic reference mm10, filter, count UMIs, single-cell 5′ end genes, and control quality per the manufacturer's parameters. Processed data were subsequently analyzed in R (version 4.0.3).

### TLS Transcriptional Signature Analysis

RNA-seq datasets were obtained from https://gdc.cancer.gov/ and https://dcc.icgc.org/repositories/ under the identifiers of TCGA-PAAD48, TCGA-BRCA49, TCTA-SKCM50, and PACA-AU15. For the TCGA-PAAD dataset, 150 patients who were histologically diagnosed as PDAC were included. Data were log-2 transformed, and known TLS gene signatures5,9,10 were extracted from each dataset. Briefly, these signatures included the genes CD79B, EIF1AY, PTGDS, CCR6, SKAP1, CETP, CD1D in Cabrita et al.5, CCL2, CCL3, CCL4, CCL5, CCL8, CCL18, CCL19, CCL21, CXCL9, CXCL10, CXCL11, and CXCL13 in Coppola et al.9, and CXCL13, CD200, FBLN7, ICOS, SGPP2, SH2D1A, TIGIT, and PDCD1 in Gu-Trantien et al.10. The signature score was calculated as the mean gene expression as described3. Pearson's correlation tests were performed using the “rcorr” function of the Hmisc package (version 4.5) in R (version 4.0.3).

### In Vitro Assays

Spleens of Il33Cit/+ mice were processed to single-cell suspension as described above. Live, CD45+, NK1.1−, CD11b+ cells were sort-purified to 90% purity using an SH800 Sony sorter (Sony Biotechnology). 5×105 myeloid cells were cultured in RPMI, 10% FBS, penicillin, streptomycin, and GM-CSF (20 ng/ml) for 72 hours at 37° C. on an ultra-low attachment 96-well plate. Cells were treated with varying concentrations of agonistic LTbR-Ig (Invitrogen) and IL33 expression was detected by flow cytometry 72 hours later.

Myeloid cells from the spleen of WT and Ltbr−/− mice were obtained as described above. 5×105 myeloid cells were cultured in RPMI, 10% FBS, penicillin, streptomycin, and GM-CSF (20 ng/ml) for 24 hours at 37° C. on an ultra-low attachment 96-well plate. Cells were treated with 0.25 μg/ml agonistic LTbR-Ig (Invitrogen) and IL33 expression was detected by flow cytometry 24 hours later.

ST2 reporter cell line activation assay: 5×104 HEK-Blue IL33 cells (Invivogen) were seeded on 96-well plate with DMEM, 10% FBS, penicillin (100 IU/ml), and streptomycin (100 μg/ml). Cells were incubated for 24 hours at 37° C. in 5% CO2 with H-rIL33 (Proteos, Inc.), H-e-rIL33, or H-e-rIL33-Fc at designated concentrations. After incubation, 20 μl of supernatant was added to 180 μl of QUANTI-Blue solution (Invivogen) per well in a flat-bottom 96-well plate. The plate was incubated for 2 hours at 37° C. in 5% CO2 followed by 630-nm wavelength absorbance detection on a Cytation 3 reader (BioTek).

Human ILC2 culture: Human ILC2s defined as live, CD45+, lineage (CD5, CD14, CD11a, CD11b, CD16, FceRIa, CD3, CD19, TCRa/b, CD56)−, CRTH2+ were sort-purified from digested human tumors, lymph nodes, and PBMCs. 1000 ILC2s were cultured in RPMI, 10% FBS, penicillin, streptomycin, human IL2 (100 IU/ml), human IL7 (10 μg/ml), and human IL15 (10 μg/ml) in U-bottom 96-well plates for 72 hours at 37° C. Cells were treated with varying concentrations of H-e-rIL33-Fc protein. To detect LT expression, the Fc portion of H-e-rIL33-Fc was blocked with anti-human IgG antibody (BD Biosciences; cat #555787); human LT was detected per the above-described staining protocol.

### Statistics

Comparisons between two groups were performed using unpaired Mann-Whitney test with the Benjamini-Krieger-Yekutieli false discovery approach for multiple time point comparisons (2-tailed). Comparisons among multiple groups were performed using 1-way ANOVA test followed by Kruskal Wallis multiple comparison post-test. Comparisons among multiple groups across multiple time points were performed using 2-way ANOVA test followed by Sidak's multiple comparison post-test. EC50 curves were compared using an extra sum of squares F test. Correlations between 2 variables were calculated using linear regression. All alpha levels were 0.05, P<0.05 was considered a significant difference. Statistical analyses were performed using R (version 4.0.3, single cell RNA sequencing) and Prism 9.2.0 (GraphPad Software, all else).

### REFERENCES

1. Sautes-Fridman, C., Petitprez, F., Calderaro, J. & Fridman, W. H. Tertiary lymphoid structures in the era of cancer immunotherapy. Nat Rev Cancer 19, 307-325 (2019).
2. Scott, I. C. et al. Interleukin-33 is activated by allergen- and necrosis-associated proteolytic activities to regulate its alarmin activity during epithelial damage. Sci Rep-uk 8, 3363 (2018).
3. Drayton, D. L., Ying, X., Lee, J., Lesslauer, W. & Ruddle, N. H. Ectopic LTαβ Directs Lymphoid Organ Neogenesis with Concomitant Expression of Peripheral Node Addressin and a HEV-restricted Sulfotransferase. J Exp Medicine 197, 1153-1163 (2003).
4. Boehm, T. & Bleul, C. C. The evolutionary history of lymphoid organs. Nat Immunol 8, 131-135 (2007).
5. Cabrita, R. et al. Tertiary lymphoid structures improve immunotherapy and survival in melanoma. Nature 577, 561-565 (2020).
6. Petitprez, F. et al. B cells are associated with survival and immunotherapy response in sarcoma. Nature 577, 556-560 (2020).
7. Helmink, B. A. et al. B cells and tertiary lymphoid structures promote immunotherapy response. Nature 577, 549-555 (2020).
8. Liu, J. et al. An Integrated TCGA Pan-Cancer Clinical Data Resource to Drive High-Quality Survival Outcome Analytics. Cell 173, 400-416.e11 (2018).
9. Coppola, D. et al. Unique Ectopic Lymph Node-Like Structures Present in Human Primary Colorectal Carcinoma Are Identified by Immune Gene Array Profiling. Am J Pathology 179, 37-45 (2011).
10. Gu-Trantien, C. et al. CD4+ follicular helper T cell infiltration predicts breast cancer survival. J Clin Invest 123, 2873-2892 (2013).
11. Goc, J. et al. Dendritic Cells in Tumor-Associated Tertiary Lymphoid Structures Signal a Th1 Cytotoxic Immune Contexture and License the Positive Prognostic Value of Infiltrating CD8+ T Cells. Cancer Res 74, 705-715 (2014).
12. Hiraoka, N. et al. Intratumoral tertiary lymphoid organ is a favourable prognosticator in patients with pancreatic cancer. British journal of cancer 112, 1782-1790 (2015).
13. Luther, S. A., Ansel, K. M. & Cyster, J. G. Overlapping Roles of CXCL13, Interleukin 7 Receptor a, and CCR7 Ligands in Lymph Node Development. J Exp Medicine 197, 1191-1198 (2003).
14. Koni, P. A. et al. Distinct Roles in Lymphoid Organogenesis for Lymphotoxins a and p Revealed in Lymphotoxin P-Deficient Mice. Immunity 6, 491-500 (1997).
15. Bailey, P. et al. Genomic analyses identify molecular subtypes of pancreatic cancer. Nature 531, 47-52 (2016).
16. Liew, F. Y., Girard, J.-P. & Turnquist, H. R. Interleukin-33 in health and disease. Nature Reviews Immunology 16, (2016).
17. Li, J. et al. Tumor Cell-Intrinsic Factors Underlie Heterogeneity of Immune Cell Infiltration and Response to Immunotherapy. Immunity 49, 178-193.e7 (2018).
18. Balachandran, V. P. et al. Identification of unique neoantigen qualities in long-term survivors of pancreatic cancer. Nature 551, 512-516 (2017).
19. Moral, J. A. et al. ILC2s amplify PD-1 blockade by activating tissue-specific cancer immunity. Nature 579, 790-6 (2020).
20. Alimzhanov, M. B. et al. Abnormal development of secondary lymphoid tissues in lymphotoxin β-deficient mice. Proc National Acad Sci 94, 9302-9307 (1997).
21. Futterer, A., Mink, K., Luz, A., Kosco-Vilbois, M. H. & Pfeffer, K. The Lymphotoxin R Receptor Controls Organogenesis and Affinity Maturation in Peripheral Lymphoid Tissues. Immunity 9, 59-70 (1998).
22. Meier, D. et al. Ectopic Lymphoid-Organ Development Occurs through Interleukin 7-Mediated Enhanced Survival of Lymphoid-Tissue-Inducer Cells. Immunity 26, 643-654 (2007).
23. Mebius, R. E., Rennert, P. & Weissman, I. L. Developing lymph nodes collect CD4+CD3−LTbeta+ cells that can differentiate to APC, NK cells, and follicular cells but not T or B cells. Immunity 7, 493-504 (1997).
24. Shikhagaie, M. M. et al. Neuropilin-1 Is Expressed on Lymphoid Tissue Residing LTi-like Group 3 Innate Lymphoid Cells and Associated with Ectopic Lymphoid Aggregates. Cell Reports 18, 1761-1773 (2017).
25. Wong, S. H. et al. Transcription factor RORα is critical for nuocyte development. Nat Immunol 13, 229-236 (2012).
26. Huang, Y. et al. IL-25-responsive, lineage-negative KLRG1(hi) cells are multipotential “inflammatory” type 2 innate lymphoid cells. Nature immunology 16, 161-169 (2015).
27. Huang, Y. et al. SiP-dependent interorgan trafficking of group 2 innate lymphoid cells supports host defense. Science (New York, NY) 359, 114-119 (2018).
28. Miller, M. M. et al. BATF acts as an essential regulator of IL-25-responsive migratory ILC2 cell fate and function. Science immunology 5, eaay3994 (2020).
29. Taylor, S. et al. PD-1 regulates KLRG1(+) group 2 innate lymphoid cells. Journal of Experimental Medicine 214, 1663-1678 (2017).
30. Ricardo-Gonzalez, R. R. et al. Tissue-specific pathways extrude activated ILC2s to disseminate type 2 immunity. Journal of Experimental Medicine 217, 171 (2020).
31. Flamar, A.-L. et al. Interleukin-33 Induces the Enzyme Tryptophan Hydroxylase 1 to Promote Inflammatory Group 2 Innate Lymphoid Cell-Mediated Immunity. Immunity (2020) doi:10.1016/j.immuni.2020.02.009.
32. Futterer, A., Mink, K., Luz, A., Kosco-Vilbois, M. H. & Pfeffer, K. The Lymphotoxin R Receptor Controls Organogenesis and Affinity Maturation in Peripheral Lymphoid Tissues. Immunity 9, 59-70 (1998).
33. Pavert, S. A. van de & Mebius, R. E. New insights into the development of lymphoid tissues. Nat Rev Immunol 10, 664-674 (2010).
34. Wege, A. K., Huber, B., Wimmer, N., Mannel, D. N. & Hehlgans, T. LTOR expression on hematopoietic cells regulates acute inflammation and influences maturation of myeloid subpopulations. Innate Immun 20, 461-470 (2013).
35. Luther, S. A., Ansel, K. M. & Cyster, J. G. Overlapping Roles of CXCL13, Interleukin 7 Receptor a, and CCR7 Ligands in Lymph Node Development. J Exp Medicine 197, 1191-1198 (2003).
36. Gunther, S. et al. IL-1 Family Cytokines Use Distinct Molecular Mechanisms to Signal through Their Shared Co-receptor. Immunity 47, 510-523.e4 (2017).
37. Sawa, S. et al. Lineage Relationship Analysis of RORγt+Innate Lymphoid Cells. Science 330, 665-669 (2010).
38. Ikeda, A. et al. Human NKp44+ Group 3 Innate Lymphoid Cells Associate with Tumor-Associated Tertiary Lymphoid Structures in Colorectal Cancer. Cancer Immunol Res 8, 724-731 (2020).
39. Peters, A. et al. Th17 cells induce ectopic lymphoid follicles in central nervous system tissue inflammation. Immunity 35, 986-96 (2011).
40. Lochner, M. et al. Microbiota-induced tertiary lymphoid tissues aggravate inflammatory disease in the absence of RORγt and LTi cells. J Exp Medicine 208, 125-134 (2010).
41. Bonilla, W. V. et al. The alarmin interleukin-33 drives protective antiviral CD8+ T cell responses. Science (New York, NY) 335, 984-989 (2012).
42. Huang, B. et al. Interleukin-33-induced expression of PIBF1 by decidual B cells protects against preterm labor. Nat Med 23, 128-135 (2016).
43. Qi, F. et al. Macrophages produce IL-33 by activating MAPK signaling pathway during RSV infection. Mol Immunol 87, 284-292 (2017).
44. Kabashima, K. et al. Intrinsic lymphotoxin-beta receptor requirement for homeostasis of lymphoid tissue dendritic cells. Immunity 22, 439-450 (2005).
45. Hung, L.-Y. et al. Cellular context of IL-33 expression dictates impact on anti-helminth immunity. Sci Immunol 5, eabc6259 (2020).
46. Zhang, M. et al. A Translocation Pathway for Vesicle-Mediated Unconventional Protein Secretion. Cell 181, 637-652.e15 (2020).47. Gao X, Wang X, Yang Q, et al. Tumoral expression of IL-33 inhibits tumor growth and modifies the tumor microenvironment through CD8+ T and NK cells. J Immunol. 2015; 194(1):438-445.
48. Kim J, Kim W, Moon U J, et al. Intratumorally Establishing Type 2 Innate Lymphoid Cells Blocks Tumor Growth. J Immunol. 2016; 196(5):2410-2423.
49. Lucarini V, Ziccheddu G, Macchia I, et al. IL-33 restricts tumor growth and inhibits pulmonary metastasis in melanoma-bearing mice through eosinophils. Oncoimmunology. 2017; 6(6):e1317420.
50. Saranchova I, Han J, Zaman R, et al. Type 2 Innate Lymphocytes Actuate Immunity Against Tumours and Limit Cancer Metastasis. Sci Rep. 2018; 8(1):2924.
51. Hollande C, Boussier J, Ziai J, et al. Inhibition of the dipeptidyl peptidase DPP4 (CD26) reveals IL-33-dependent eosinophil-mediated control of tumor growth. Nat Immunol. 2019; 20(3):257-264.
52. Eissmann M F, Dijkstra C, Wouters M A, et al. Interleukin 33 Signaling Restrains Sporadic Colon Cancer in an Interferon-gamma-Dependent Manner. Cancer immunology research. 2018; 6(4):409-421.
53. Qin L, Dominguez D, Chen S, et al. Exogenous IL-33 overcomes T cell tolerance in murine acute myeloid leukemia. Oncotarget. 2016; 7(38):61069-61080.
54. Nam K T, Lee H J, Mok H, et al. Amphiregulin-deficient mice develop spasmolytic polypeptide expressing metaplasia and intestinal metaplasia. Gastroenterology. 2009; 136(4):1288-1296.
55. Sekiya A, Suzuki S, Tanaka A, et al. Interleukin33 expression in ovarian cancer and its possible suppression of peritoneal carcinomatosis. Int J Oncol. 2019; 55(3):755-765.
56. Perales-Puchalt A, Svoronos N, Villarreal D O, et al. IL-33 delays metastatic peritoneal cancer progression inducing an allergic microenvironment. Oncoimmunology. 2019; 8(1):e1515058.
57. WO2021/003138A1; Memorial Sloan Kettering Cancer Center; Balachandran et al.

Methods and Compositions for Treatment of Pancreatic Cancer.

1. A method of inducing the formation of de novo tertiary lymphoid structures in a subject, the method comprising administering to the subject an effective amount of an IL33 protein.

2. The method of claim 1, wherein the IL33 protein is human IL33.

3. The method of claim 1, wherein the IL33 protein is mature human IL33.

4. The method of claim 1, wherein the IL33 protein is a mature human IL33 protein having the sequence of SEQ ID NO. 1.

5. The method of claim 1, wherein the subject is a human.

6. The method of claim 1, wherein the subject has cancer.

7. The method of claim 1, wherein the subject has pancreatic cancer, breast cancer or melanoma.

8. The method of claim 1, wherein the subject has pancreatic ductal adenocarcinoma (PDAC).

9. The method of claim 1, wherein the subject has a cancer that resistant to PD-1 and/or PD-L1 inhibitor treatment.

10. The method of claim 1, wherein subject has a chronic inflammatory condition.

11. The method of claim 1, wherein the subject has a chronic inflammatory gastrointestinal condition.

12. The method of claim 1, wherein the subject has colitis, ulcerative colitis, inflammatory bowel disease, irritable bowel syndrome or Crohn's disease.
